(19) United States
(12) Patent Application Publication
(10) Pub. No.: US 2022/0185863 A1
(43) Pub. Date: Jun. 16, 2022

(54) COMBINATION THERAPIES

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US)

(21) Appl. No.: 17/433,692

(22) PCT Filed: Feb. 27, 2020
(86) PCT No.: PCT/US2020/020076

§ 371 Date: Aug. 25, 2021

Related U.S. Application Data
(60) Provisional application No. 62/894,479, filed on Aug. 30, 2019.
(60) Provisional application No. 62/811,861, filed on Feb. 28, 2019.

(51) Int. Cl. C07K 14/725; C07K 14/705; A61P 37/04

(52) U.S. Cl. CPC C07K 14/7051; A61P 37/04; C07K 14/70575

(57) ABSTRACT

The present invention relates to, inter alia, combinations of compositions which include chimeric proteins that find use in methods for treating disease, such as immunotherapies for cancer and autoimmunity.

### PRIORITY

This application claims the benefit of, and priority to, US Application Nos. 62/811,861, filed Feb. 28, 2019, and 62/894,479, filed Aug. 30, 2019, each of which is hereby incorporated by reference in its entirety.

### TECHNICAL FIELD

The present invention relates to, inter alia, combinations of compositions which include chimeric proteins that find use in methods for treating disease, such as immunotherapies for cancer and autoimmunity.

### DESCRIPTION OF THE TEXT FILE SUBMITTED ELECTRONICALLY

This application contains a sequence listing. It has been submitted electronically via EFS-Web as an ASCII text file entitled “SHK-014PC_SequenceListing_ST25”. The sequence listing is 45,056 bytes in size, and was created on Feb. 27, 2020. The sequence listing is hereby incorporated by reference in its entirety.

### BACKGROUND

The immune system is central to the body's response to cancer cells and disease-causing foreign entities.

Many cancers, however, have developed mechanisms to avoid the immune system by, for instance, delivering or propagating immune inhibitory signals. Additionally, many anti-cancer therapeutics do not directly stimulate and/or activate the immune response. Current combination immunotherapy with bispecific antibodies, linked scFv's, or T cell engagers have not been able to both block checkpoints (immune inhibitory signals) and agonize (stimulate) TNF receptors. This is likely because these molecules lose target avidity when engineered to bind multiple targets with monovalent antigen binding arms. Thus, there remains a need to develop therapeutics that, at least, are endowed with multiple functionalities but still retain target avidity—for instance, reverse immune inhibitory signals and stimulating an anti-cancer immune response.

### SUMMARY

Accordingly, in various aspects, the present invention provides compositions and methods that are useful for cancer immunotherapy. For instance, the present invention, in part, relates to methods for treating cancer comprising administering (either simultaneously or sequentially) at least one antibody directed to an immune checkpoint molecule; a stimulator of interferon genes (STING) agonist; and/or one or more chimeric proteins, in which each chimeric protein is capable of blocking immune inhibitory signals and/or stimulating immune activating signals.

An aspect of the present invention relates to a method for treating a cancer in a subject in need thereof comprising: providing the subject a first pharmaceutical composition comprising an antibody that is capable of binding cytotoxic T lymphocyte-associated antigen 4 (CTLA-4) and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

Another aspect of the present invention relates to a method for treating a cancer in a subject who has undergone or is undergoing treatment with an antibody that is capable of binding cytotoxic T lymphocyte-associated antigen 4 (CTLA-4). The method comprises providing the subject a pharmaceutical composition comprising a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

Yet another aspect of the present invention provides a method for treating a cancer in a subject comprising providing the subject a pharmaceutical composition comprising an antibody that is capable of binding cytotoxic T lymphocyte-associated antigen 4 (CTLA-4). Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

An aspect of the present invention relates to a method for treating a cancer in a subject in need thereof comprising: providing the subject a first pharmaceutical composition comprising an antibody that is capable of binding programmed cell death protein 1 (PD-1) or binding a PD-1 ligand and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

Another aspect of the present invention relates to a method for treating a cancer in a subject who has undergone or is undergoing treatment with an antibody that is capable of binding programmed cell death protein 1 (PD-1) or binding a PD-1 ligand. The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

Yet another aspect of the present invention provides a method for treating a cancer in a subject comprising providing the subject a pharmaceutical composition comprising an antibody that is capable of binding programmed cell death protein 1 (PD-1) or binding a PD-1 ligand. Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

An aspect of the present invention relates to a method for treating a cancer in a subject in need thereof comprising: providing the subject a first pharmaceutical composition comprising a stimulator of interferon genes (STING) agonist and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

Another aspect of the present invention relates to a method for treating a cancer in a subject who has undergone or is undergoing treatment with a stimulator of interferon genes (STING) agonist. The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

Yet another aspect of the present invention provides a method for treating a cancer in a subject comprising providing the subject a pharmaceutical composition comprising a stimulator of interferon genes (STING) agonist. Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

An aspect of the present invention relates to a method for treating a cancer in a subject in need thereof comprising: providing the subject a first pharmaceutical composition comprising an antibody that is capable of binding programmed cell death protein 1 (PD-1) or binding a PD-1 ligand and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of LIGHT, wherein the portion is capable of binding a LIGHT receptor, and (c) a linker linking the first domain and the second domain.

Another aspect of the present invention relates to a method for treating a cancer in a subject who has undergone or is undergoing treatment with an antibody that is capable of binding programmed cell death protein 1 (PD-1) or binding a PD-1 ligand. The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of LIGHT, wherein the portion is capable of binding a LIGHT receptor, and (c) a linker linking the first domain and the second domain.

Yet another aspect of the present invention provides a method for treating a cancer in a subject comprising providing the subject a pharmaceutical composition comprising an antibody that is capable of binding programmed cell death protein 1 (PD-1) or binding a PD-1 ligand. Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of LIGHT, wherein the portion is capable of binding a LIGHT receptor, and (c) a linker linking the first domain and the second domain.

Yet another aspect of the present invention provides a method for evaluating the efficacy of cancer treatment in a subject in need thereof, wherein the subject is suffering from a cancer, the method comprising the steps of (i) providing the subject a pharmaceutical composition comprising a heterologous chimeric protein comprising: (A) a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain; and (B) an anti-immune checkpoint antibody; (ii) obtaining a biological sample from the subject; (iii) performing an assay on the biological sample to determine level and/or activity of a mononuclear cells; and (iv) continuing administration of the heterologous chimeric protein if the subject has an increase in the level and/or activity of CD4⁺ cells, CD8⁺ T cells, and/or NKP46⁺ NK cells.

Yet another aspect of the present invention provides method for evaluating the efficacy of cancer treatment in a subject in need thereof, wherein the subject is suffering from a cancer, the method comprising the steps of (i) providing the subject a pharmaceutical composition comprising (A) a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain; and (B) an anti-immune checkpoint antibody; (ii) obtaining a biological sample from the subject; (iii) performing an assay on the biological sample to determine level and/or activity of a mononuclear cells; and (iv) continuing administration of the heterologous chimeric protein if the subject has an increase in the level and/or activity of CD4⁺ T cells, CD8⁺ T cells, and/or NKP46⁺ NK cells.

Any aspect or embodiment disclosed herein can be combined with any other aspect or embodiment as disclosed herein.

### BRIEF DESCRIPTION OF THE DRAWINGS

FIG. 1A to FIG. 1D show schematic illustrations of Type I transmembrane proteins (FIG. 1A and FIG. 1B, left proteins) and Type II transmembrane proteins (FIG. 1A and FIG. 1B, right proteins). A Type I transmembrane protein and a Type II transmembrane protein may be engineered such that their transmembrane and intracellular domains are omitted and the transmembrane proteins' extracellular domains are adjoined using a linker sequence to generate a single chimeric protein. As shown in FIG. 1C and FIG. 1D, the extracellular domain of a Type I transmembrane protein, e.g., PD-1, SIRPα(CD172a), TIGIT, and TIM-3, and the extracellular domain of a Type II transmembrane protein, e.g., 4-1BBL, CD40L, GITRL, and OX40L, are combined into a single chimeric protein. FIG. 1C depicts the linkage of the Type I transmembrane protein and the Type II transmembrane protein by omission of the transmembrane and intracellular domains of each protein, and where the liberated extracellular domains from each protein have been adjoined by a linker sequence. The extracellular domains in this depiction may include the entire amino acid sequence of the Type I protein (e.g., PD-1, SIRPα(CD172a), TIGIT, and TIM-3,) and/or Type II protein (e.g., 4-1BBL, CD40L, GITRL, and OX40L) which is typically localized outside the cell membrane, or any portion thereof which retains binding to the intended receptor or ligand. Moreover, the chimeric protein used in a method of the present invention comprises sufficient overall flexibility and/or physical distance between domains such that a first extracellular domain (shown at the left end of the chimeric protein in FIG. 1C and FIG. 1D) is sterically capable of binding its receptor/ligand and/or a second extracellular domain (shown at the right end of the chimeric protein in FIG. 1C and FIG. 1D) is sterically capable of binding its receptor/ligand. FIG. 1D depicts adjoined extracellular domains in a linear chimeric protein wherein each extracellular domain of the chimeric protein is facing “outward”.

FIG. 2 shows immune inhibitory and immune stimulatory signaling that is relevant to the present invention (from Mahoney, Nature Reviews Drug Discovery 2015:14; 561-585).

FIG. 3A shows in vivo reductions in tumor volume size resulting from the methods of cancer treatments according to the present invention. FIG. 3B shows Kaplan-Meier plots of the percent survival days after tumor inoculation for the different combinations shown in FIG. 3A. In these figures, the term “ARC” refers to the TIGIT-Fc-OX40L chimeric protein. FIG. 3C includes data relevant to the graphs of FIG. 3A and FIG. 3B.

FIG. 4A shows in vivo reductions in tumor volume size resulting from the methods of cancer treatments according to the present invention. FIG. 4B shows Kaplan-Meier plots of the percent survival days after tumor inoculation for the different antibody combinations shown in FIG. 4A. In these figures, the term “ARC” refers to the TIGIT-Fc-OX40L chimeric protein. FIG. 4C includes data relevant to the graphs of FIG. 4A and FIG. 4B.

FIG. 5A shows tumor growth kinetics in a mouse challenged with CT26 tumor and treated as indicated in the legend (at day 10, the order of curves, top to bottom, is vehicle, anti-PD1, TIGIT-Fc-LIGHT, TIGIT-Fc-LIGHT+anti-PD1). FIG. 5B is a Kaplan Meyer plot of survival and statistics of the CT26 tumor experiment of FIG. 5A. FIG. 5C and FIG. 5D include data relevant to the graphs of FIG. 5A and FIG. 5B.

FIG. 6A to FIG. 6G show the results of immune phenotyping of the tumor infiltrating lymphocytes (TIL) in BALB/C mice harboring CT26 (colorectal carcinoma) tumor allografts treated with ARC (TIGIT-Fc-OX40L or TIGIT-Fc-LIGHT) alone or the combination with anti-PD-1 (clone RMP1-14) antibody. The fractions (expressed as percentages) of total CD8⁺ cells (FIG. 6A), total Perforin CD8⁺ cells (FIG. 6B), total IFNγ⁺ CD8⁺ cells (FIG. 6C), total AH1-tetramer CD8⁺ cells (antigen-specific CD8⁺ cells) (FIG. 6D), total CD4±cells (FIG. 6E), total NKP46⁺ NK cells (FIG. 6F) and total IFNγ±(NKP46⁺ NK) cells (FIG. 6G) in the indicated cell populations in the dissociated tumor tissue as determined by flow cytometry analyses.

### DETAILED DESCRIPTION

The present invention is based, in part, on the discovery of methods for treating cancer comprising administering (either simultaneously or sequentially) at least one antibody directed to an immune checkpoint molecule; a stimulator of interferon genes (STING) agonist; and/or one or more chimeric proteins, in which each chimeric protein is capable of blocking immune inhibitory signals and/or stimulating immune activating signals.

Importantly, since the antibodies directed to immune checkpoint molecules; STING agonists; and/or chimeric proteins used in methods of the present invention disrupt, block, reduces, inhibit, and/or sequester the transmission of immune inhibitory signals, e.g., originating from a cancer cell that is attempting to avoid its detection and/or destruction and/or enhance, increase, and/or stimulate the transmission of an immune stimulatory signal to an anti-cancer immune cell, the methods can provide an anti-tumor effect by multiple distinct pathways. By treating cancer via multiple distinct pathways, the methods of the present invention are more likely to provide any anti-tumor effect in a patient and/or to provide an enhanced anti-tumor effect in a patient. Moreover, since the methods operate by multiple distinct pathways, they can be efficacious, at least, in patients who do not respond, respond poorly, or become resistant to treatments that target one of the pathways. Thus, a patient who is a poor responder to treatments acting via one of the two pathways, can receive a therapeutic benefit by targeting multiple pathways.

Antibodies

The methods of the present invention comprise methods for treating cancer, which in embodiments, comprise administering an immunotherapy comprising an antibody capable of binding an immune checkpoint molecule.

The antibody may be selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In embodiments, the antibody is a monoclonal antibody, e.g., a humanized monoclonal antibody. Accordingly, as used herein, the term “antibody” includes a monoclonal antibody (e.g., a humanized monoclonal antibody), a polyclonal antibody, an antibody fragment, a Fab, a Fab′, a Fab′-SH, a F(ab′)2, an Fv, a single chain Fv, a diabody, a linear antibody, a bispecific antibody, a multispecific antibody, a chimeric antibody, a humanized antibody, a human antibody, or a fusion protein comprising the antigen-binding portion of an antibody.

In embodiments, the antibody is capable of binding CTLA-4. Illustrative antibodies capable of binding CTLA-4 include YERVOY (ipilimumab), 9D9, tremelimumab (formerly ticilimumab, CP-675,206; MedImmune), AGEN1884, and RG2077.

In embodiments, the antibody is capable of binding PD-1 or a PD-1 ligand. Illustrative antibodies capable of binding PD-1 or a PD-1 ligand include nivolumab (ONO 4538, BMS 936558, MDX1106, OPDIVO (Bristol Myers Squibb)), pembrolizumab (KEYTRUDA/MK 3475, Merck), and cemiplimab ((REGN-2810). Such an antibody is capable of inhibiting the interaction of PD-1 with one or more of its ligands.

STING Agonists

The methods of the present invention comprise methods for treating cancer, which in embodiments, comprise administering a pharmaceutical composition comprising a stimulator of interferon genes (STING) agonist. The STING pathway is known to turn on an interferon response which attracts immune cells. Without wishing to be bound by theory, turning on the STING pathway, via a STING agonist, would result in immune activation and stimulation of immune cells to attack a cancer.

In embodiments, the STING Agonist is selected from the group consisting of 5,6-dimethylxanthenone-4-acetic acid (DMXAA), MIW815(ADU-S100), CRD5500, MK-1454, SB11285, IMSA101, and any STING agonist described in US20140341976, US20180028553, US20180230178, U.S. Pat. No. 9,549,944, WO2015185565, WO2016120305, WO2017044622, WO2017027645, WO2017027646, WO2017093933, WO2017106740, WO2017123657, WO2017123669, WO2017161349, WO2017175147, WO2017175156, WO2017176812, WO2018009466, WO2018045204, WO2018060323, WO2018098203, WO2018100558, WO2018138684, WO2018138685, WO2018152450, WO2018152453, WO2018172206, WO2018198084, WO2018234805, WO2018234807, WO2018234808, WO2019023459, WO2019046496, WO2019046498, WO2019046500, WO2019074887, WO2019079261, WO2019118839, WO2019125974, or WO2019160884, the contents of which are incorporated herein by reference in their entireties.

Chimeric Proteins

The methods of the present invention comprise methods for treating cancer, which in embodiments, comprise administering a pharmaceutical composition comprising a chimeric protein capable of blocking immune inhibitory signals and/or stimulating immune activating signals.

Chimeric proteins used in methods of the present invention comprise a general structure of: N terminus-(a)-(b)-(c)-C terminus, where (a) is a first domain comprising an extracellular domain of Type I transmembrane protein, (b) is a linker adjoining the first domain and the second domain, e.g., the linker comprising at least one cysteine residue capable of forming a disulfide bond and/or comprising a hinge-CH2-CH3 Fc domain, and (c) is a second domain comprising an extracellular domain of a Type II transmembrane protein; wherein the linker connects the first domain and the second domain. Alternately, a chimeric proteins used in methods of the present invention comprise a general structure of: N terminus-(a)-(b)-(c)-C terminus, where (a) is a first domain comprising an extracellular domain of Type I transmembrane protein, (b) is a linker adjoining the first domain and the second domain, e.g., the linker comprising at least one cysteine residue capable of forming a disulfide bond and/or comprising a hinge-CH2-CH3 Fc domain, and (c) is a second domain comprising an extracellular domain of another Type I transmembrane protein; wherein the linker connects the first domain and the second domain.

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell) in the extracellular environment. Without wishing to be bound by theory, the trans-membrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa).

In embodiments, an extracellular domain refers to a portion of a transmembrane protein which is sufficient for binding to a ligand or receptor and is effective in transmitting a signal to a cell. In embodiments, an extracellular domain is the entire amino acid sequence of a transmembrane protein which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

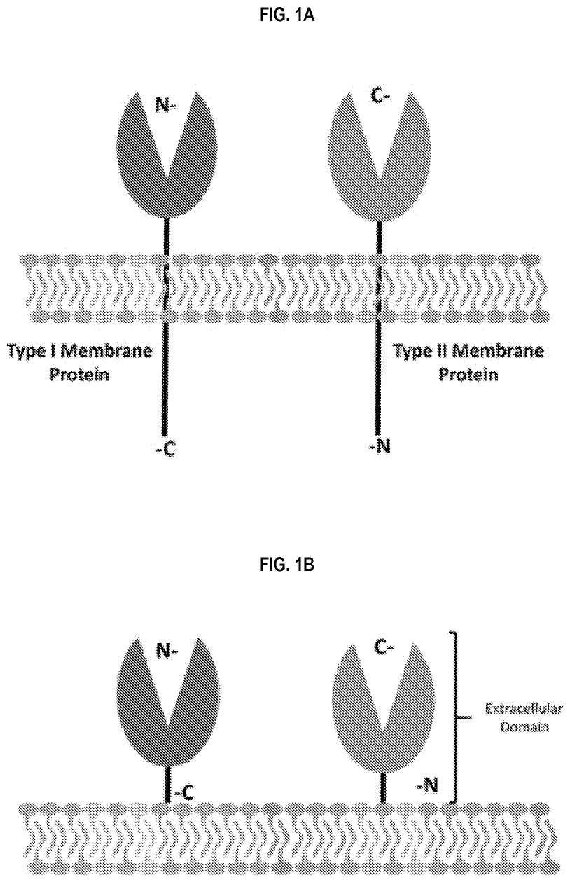

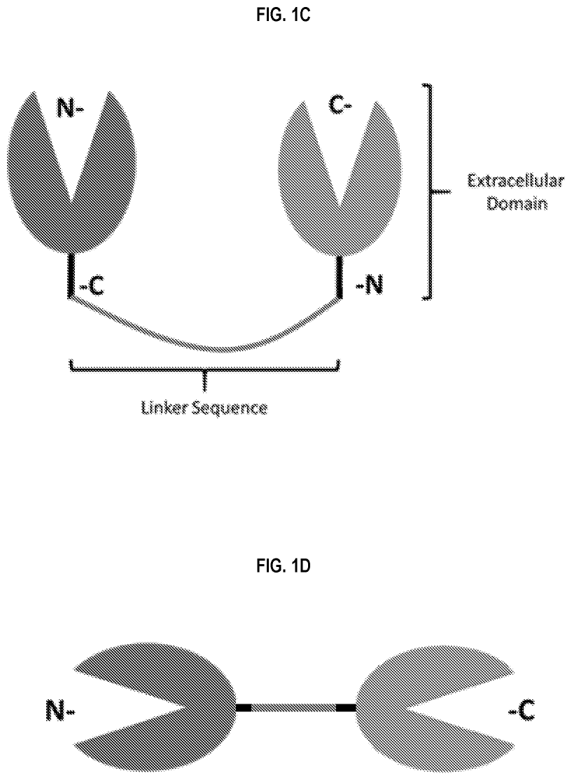

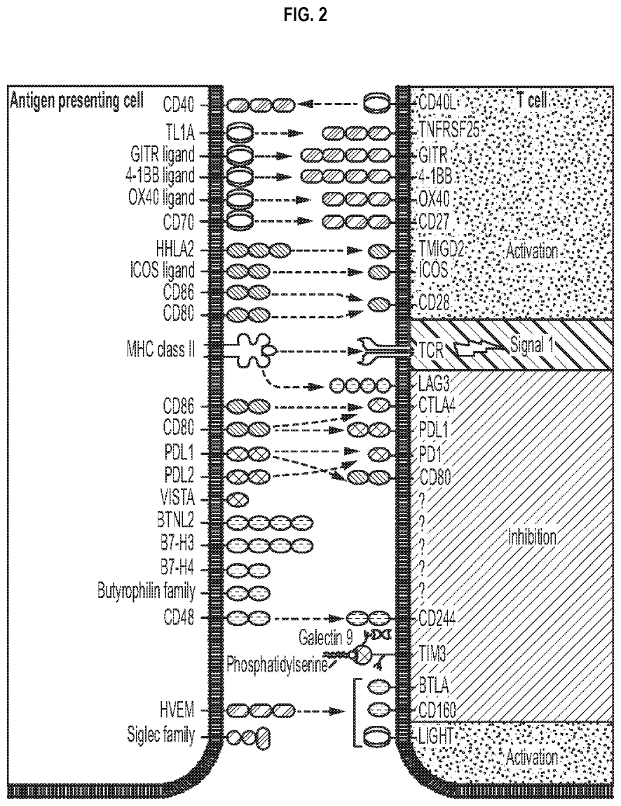

There are generally two types of single-pass transmembrane proteins: Type I transmembrane proteins which have an extracellular amino terminus and an intracellular carboxy terminus (see, FIG. 1A, left protein) and Type II transmembrane proteins which have an extracellular carboxy terminus and an intracellular amino terminus (see, FIG. 1A, right protein). Type I and Type II transmembrane proteins can be either receptors or ligands. For Type I transmembrane proteins (e.g., PD-1, SIRPα(CD172a), TIGIT, and TIM-3), the amino terminus of the protein faces outside the cell, and therefore contains the functional domains that are responsible for interacting with other binding partners (either ligands or receptors) in the extracellular environment (see, FIG. 1B, left protein). For Type II transmembrane proteins (e.g., 4-1BBL, CD40L, GITRL, and OX40L), the carboxy terminus of the protein faces outside the cell, and therefore contains the functional domains that are responsible for interacting with other binding partners (either ligands or receptors) in the extracellular environment (see, FIG. 1B, right protein). Thus, these two types of transmembrane proteins have opposite orientations to each other relative to the cell membrane.

Chimeric proteins used in methods of the present invention comprise an extracellular domain of a Type I transmembrane protein selected from PD-1, SIRPα(CD172a), TIGIT, and TIM-3 and an extracellular domain of a Type II transmembrane protein selected from 4-1BBL, CD40L, GITRL, and OX40L. Thus, a chimeric protein used in a method of the present invention comprises, at least, a first domain comprising the extracellular domain of PD-1, SIRPα(CD172a), TIGIT, or TIM-3, which is connected—directly or via a linker—to a second domain comprising the extracellular domain of 4-1BBL, CD40L, GITRL, or OX40L. As illustrated in FIG. 1C and FIG. 1D, when the domains are linked in an amino-terminal to carboxy-terminal orientation, the first domain is located on the “left” side of the chimeric protein and is “outward facing” and the second domain is located on “right” side of the chimeric protein and is “outward facing”.

Other configurations of first and second domains are envisioned, e.g., the first domain is inward facing and the second domain is outward facing, the first domain is outward facing and the second domain is inward facing, and the first and second domains are both inward facing. When both domains are “inward facing”, the chimeric protein would have an amino-terminal to carboxy-terminal configuration comprising an extracellular domain of a Type II transmembrane protein, a linker, and an extracellular domain of Type I transmembrane protein. In such configurations, it may be necessary for the chimeric protein to include extra “slack”, as described elsewhere herein, to permit binding domains of the chimeric protein to one or both of its receptors/ligands.

In embodiments, the extracellular domain of a Type I transmembrane protein is from TIGIT.

TIGIT is a poliovirus receptor (PVR)-like protein, an immunoreceptor expressed on T cells that contains immunoglobulin and immunoreceptor tyrosine-based inhibitory motif (ITIM) domains. As such, TIGIT acts as an inhibitory immune checkpoint on both T cells and natural killer (NK) cells, providing an opportunity to target both the adaptive and innate arms of the immune system.

TIGIT is expressed on NK cells and subsets of activated, memory and regulatory T cells, and particularly on follicular helper T cells within secondary lymphoid organs CD155/PVR is up-regulated on endothelial cells by IFN-gamma and is highly expressed on immature thymocytes, lymph node dendritic cells, and tumor cells of epithelial and neuronal origin. In embodiments, the present chimeric proteins (e.g., comprising the TIGIT extracellular domain) modulate any of the cells described immediately above (e.g., in the context of an immune synapse).

TIGIT binds CD155/PVR, Nectin-2, Nectin-3 and Nectin-4. In embodiments, the present chimeric proteins (e.g., comprising the TIGIT extracellular domain) modulate the binding of TIGIT to CD155/PVR (e.g., reduce or disrupt the binding or signal transmission). In embodiments, the present chimeric proteins (e.g., comprising the TIGIT extracellular domain) modulate the binding of TIGIT to Nectin-2 (e.g., reduce or disrupt the binding or signal transmission). In embodiments, the present chimeric proteins (e.g., comprising the TIGIT extracellular domain) modulate the binding of TIGIT to Nectin-3 (e.g., reduce or disrupt the binding or signal transmission). In embodiments, the present chimeric proteins (e.g., comprising the TIGIT extracellular domain) modulate the binding of TIGIT to Nectin-4 (e.g., reduce or disrupt the binding or signal transmission).

In aspects, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, a heterologous chimeric protein comprises a first domain which comprises substantially all of the extracellular domain of TIGIT and/or the second domain which comprises substantially all of the extracellular domain of OX40L. In embodiments, the first domain which comprises substantially all of the extracellular domain of TIGIT. In embodiments, the second domain which comprises substantially all of the extracellular domain of OX40L.

In embodiments, a chimeric protein used in methods of the present invention comprises a portion of the extracellular domain of human TIGIT which comprises the following amino acid sequence:

(SEQ ID NO: 57)
MMTGTIETTGNISAEKGGSIILQCHLSSTTAQVTQVNWEQQDQLLAICN
ADLGWHISPSFKDRVAPGPGLGLTLQSLTVNDTGEYFCIYHTYPDGTYT
GRIFLEVLESSVAEHGARFQIP

In embodiments, a chimeric protein used in methods of the present invention comprises a variant of the extracellular domain of TIGIT. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

One of ordinary skill may select variants of the known amino acid sequence of TIGIT by consulting the literature, e.g., Stanietsky et al., “The interaction of TIGIT with PVR and PVRL2 inhibits human NK cell cytotoxicity.” PNAS U.S.A. 106 (42), 17858-17863 (2009); Boles et al., “A novel molecular interaction for the adhesion of follicular CD4 T cells to follicular DC.” Eur. J. Immunol. 39 (3), 695-703 (2009); Yu et al., “The surface protein TIGIT suppresses T cell activation by promoting the generation of mature immunoregulatory dendritic cells.” Nat. Immunol. 10 (1), 48-57 (2009); and Levin et al., “Vstm3 is a member of the CD28 family and an important modulator of T-cell function.” Eur. J. Immunol. 41 (4), 902-915 (2011); each of which is incorporated by reference in its entirety.

In embodiments, the extracellular domain of a Type II transmembrane protein is from OX40L.

In embodiments, a chimeric protein used in methods of the present invention comprises the extracellular domain of human OX40L which comprises the following amino acid sequence:

(SEQ ID NO: 58)
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL

In embodiments, a chimeric protein used in methods of the present invention comprises a variant of the extracellular domain of OX40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

One of ordinary skill may select variants of the known amino acid sequence of OX40L by consulting the literature, e.g., Godfrey et al., “Identification of a human OX-40 ligand, a costimulator of CD4+ T cells with homology to tumor necrosis factor.” J. Exp. Med. 180 (2), 757-762 (1994); Baum et al., “Molecular characterization of murine and human OX40/OX40 ligand systems: identification of a human OX40 ligand as the HTLV-1-regulated protein gp34.” EMBO J. 13 (17), 3992-4001 (1994); Ohshima et al., “Expression and function of OX40 ligand on human dendritic cells.” J. Immunol. 159 (8), 3838-3848 (1997); and Croft “Control of immunity by the TNFR-related molecule OX40 (CD134).” Annu. Rev. Immunol. 28, 57-78 (2010), each of which is incorporated by reference in its entirety.

In embodiments, the extracellular domain of a Type II transmembrane protein is from LIGHT.

In embodiments, a chimeric protein used in methods of the present invention comprises the extracellular domain of human LIGHT which comprises the following amino acid sequence:

(SEQ ID NO: 59)
LQLHWRLGEMVTRLPDGPAGSWEQLIQERRSHEVNPAAHLTGANSSLTG
SGGPLLWETQLGLAFLRGLSYHDGALVVTKAGYYYIYSKVQLGGVGCPL
GLASTITHGLYKRTPRYPEELELLVSQQSPCGRATSSSRVWWDSSFLGG
VVHLEAGEKWVRVLDERLVRLRDGTRSYFGAFMV

In embodiments, a chimeric protein used in methods of the present invention comprises a variant of the extracellular domain of LIGHT. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

One of ordinary skill may select variants of the known amino acid sequence of LIGHT by consulting the literature, e.g., Mauri, et al., “LIGHT, a new member of the TNF superfamily, and lymphotoxin alpha are ligands for herpesvirus entry mediator.” Immunity 8 (1), 21-30 (1998); Tamada et al., “LIGHT, a TNF-like molecule, costimulates T cell proliferation and is required for dendritic cell-mediated allogeneic T cell response.” J. Immunol. 164 (8), 4105-4110 (2000); Liu et al., “Mechanistic basis for functional promiscuity in the TNF and TNF receptor superfamilies: structure of the LIGHT:DcR3 assembly” Structure 22 1252-62 (2014); Faustman et al., “Structural principles of tumor necrosis factor superfamily signaling.” Sci Signal 11 (2018); Sudhamsu et al., “Dimerization of LTI3R by LTα1β2 is necessary and sufficient for signal transduction” Proc. Natl. Acad. Sci. U.S.A. 110 19896-19901 (2013); Savvides et al., “Mechanisms of immunomodulation by mammalian and viral decoy receptors: insights from structures. Felix J, SN. Nat Rev Immunol 17 112-129 (2017)”; Ward-Kavanagh et al., “The TNF Receptor Superfamily in Co-stimulating and Co-inhibitory Responses.” Immunity 44 1005-1019 (2016); and Wajant “Principles of antibody-mediated TNF receptor activation.” Cell Death Differ 22 1727-1741 (2015), each of which is incorporated by reference in its entirety.

In embodiments, the chimeric protein of the present invention and/or chimeric protein used in methods of the present invention comprises the hinge-CH2-CH3 domain from a human IgG4 antibody sequence (SEQ ID NO: 1, SEQ ID NO: 2, or SEQ ID NO: 3).

In embodiments, the chimeric protein of the present invention and/or chimeric protein used in methods of the present invention comprises an extracellular domain of TIGIT and the extracellular domain of OX40L, using the hinge-CH2-CH3 domain from a human IgG4 antibody sequence as a linker. In embodiments, the, so-called, TIGIT-Fc-OX40L chimeric protein comprises the following amino acid sequence:

(SEQ ID NO: 60)
MMTGTIETTGNISAEKGGSIILQCHLSSTTAQVTQVNWEQQDQLLAICN
ADLGWHISPSFKDRVAPGPGLGLTLQSLTVNDTGEYFCIYHTYPDGTYT
GRIFLEVLESSVAEHGARFQIPSKYGPPCPPCPAPEFLGGPSVFLFPPK
PKDQLMISRTPEVTCVVVDVSQEDPEVQFNWYVDGVEVHNAKTKPREEQ
FNSTYRWSVLTVLHQDWLSGKEYKCKVSSKGLPSSIEKTISNATGQPRE
PQVYTLPPSQEEMTKNQVSLTCLVKGFYPSDIAVEWESNGQPENNYKTT
PPVLDSDGSFFLYSRLTVDKSRWQEGNVFSCSVLHEALHNHYTQKSLSL
SLGKIEGRMDQVSHRYPRIQSIKVQFTEYKKEKGFILTSQKEDEIMKVQ
NNSVIINCDGFYLISLKGYFSQEVNISLHYQKDEEPLFQLKKVRSVNSL
MVASLTYKDKVYLNVTTDNTSLDDFHVNGGELILIHQNPGEFCVL

In embodiments, the present chimeric proteins may be variants described herein, for instance, the present chimeric proteins may have a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the present chimeric proteins, e.g., one or more of SEQ ID NO: 60.

In embodiments, the chimeric protein of the present invention and/or chimeric protein used in methods of the present invention comprises an extracellular domain of TIGIT and the extracellular domain of LIGHT, using the hinge-CH2-CH3 domain from a human IgG4 antibody sequence as a linker. In embodiments, the, so-called, TIGIT-Fc-LIGHT chimeric protein comprises the following amino acid sequence:

(SEQ ID NO: 61)
MEWSVWFLFFLSVTTGVHSMMTGTIETTGNISAEKGGSIILQCHLSSTT
AQVTQVNWEQQDQLLAICNADLGWHISPSFKDRVAPGPGLGLTLQSLIV
NDTGEYFCIYHTYPDGTYTGRIFLEVLESSVAEHGARFQIPSKYGPPCP
PCPAPEFLGGPSVFLFPPKPKDQLMISRTPEVTCVVVDVSQEDPEVQFN
WYVDGVEVHNAKTKPREEQFNSTYRVVSVLTVLHQDWLSGKEYKCKVSS
KGLPSSIEKTISNATGQPREPQVYTLPPSQEEMTKNQVSLTCLVKGFYP
SDIAVEWESNGQPENNYKTTPPVLDSDGSFFLYSRLTVDKSRWQEGNVF
SCSVLHEALHNHYTQKSLSLSLGKIEGRMDLQLHWRLGEMVTRLPDGPA
GSWEQLIQERRSHEVNPAAHLTGANSSLTGSGGPLLWETQLGLAFLRGL
SYHDGALWTKAGYYYIYSKVQLGGVGCPLGLASTITHGLYKRTPRYPEE
LELLVSQQSPCGRATSSSRVWWDSSFLGGVVHLEAGEKVWRVLDERLVR
LRDGTRSYFGAFMV.

In embodiments, the present chimeric proteins may be variants described herein, for instance, the present chimeric proteins may have a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the present chimeric proteins, e.g., one or more of SEQ ID NO: 61.

In any herein-disclosed aspect and embodiment, the chimeric protein may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, based on similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine 3-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as 8 methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, a chimeric protein is capable of binding murine ligand(s)/receptor(s).

In embodiments, a chimeric protein is capable of binding human ligand(s)/receptor(s).

In embodiments, each extracellular domain (or variant thereof) of the chimeric protein binds to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the chimeric protein binds to its cognate receptor or ligand with a K_{D} of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CSF1 with a K_{D} of less than about 1 nM, about 900 μM, about 800 μM, about 700 μM, about 600 μM, about 500 μM, about 400 μM, about 300 μM, about 200 μM, about 100 μM, about 90 μM, about 80 μM, about 70 μM, about 60 μM about 55 μM about 50 μM about 45 μM, about 40 μM, about 35 μM, about 30 μM, about 25 μM, about 20 μM, about 15 μM, or about 10 μM, or about 1 μM (as measured, for example, by surface plasmon resonance or biolayer interferometry).

As used herein, a variant of an extracellular domain is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether. In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, s/he would know which mutations would permit binding and which would eliminate binding.

In embodiments, the chimeric protein exhibits enhanced stability, high-avidity binding characteristics, prolonged off-rate for target binding and protein half-life relative to single-domain fusion protein or antibody controls.

A chimeric protein used in a method of the present invention may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein. Alternately, a second extracellular domain may be directly linked (e.g., via a peptide bond) to a third extracellular domain. In embodiments, a chimeric protein includes extracellular domains that are directly linked and extracellular domains that are indirectly linked via a linker, as disclosed herein.

Chimeric proteins of the present invention and/or chimeric proteins used in methods of the present invention have a first domain which is sterically capable of binding its ligand/receptor and/or a second domain which is sterically capable of binding its ligand/receptor. This means that there is sufficient overall flexibility in the chimeric protein and/or physical distance between an extracellular domain (or a portion thereof) and the rest of the chimeric protein such that the ligand/receptor binding domain of the extracellular domain is not sterically hindered from binding its ligand/receptor. This flexibility and/or physical distance (which is herein referred to as “slack”) may be normally present in the extracellular domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance.

Chimeric proteins described one or more of WO2018/157162; WO2018/157165; WO2018/157164; WO2018/157163; and WO2017/059168 may be used, without limitation, in the present invention. The contents of each of which is incorporated herein by reference in its entirety.

Linkers

In embodiments, the chimeric protein used in a method of the present invention comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

Importantly, inter alia, stabilization in a linker region including one or more disulfide bonds provides for improved chimeric proteins that can maintain a stable and producible multimeric state.

In a chimeric protein used in a method of the present invention, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present invention comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein used in a method of the present invention, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NO: 4 to SEQ ID NO: 50 (or a variant thereof). In embodiments, the linker comprises two or more joining linkers each joining linker independently selected from SEQ ID NO: 4 to SEQ ID NO: 50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the chimeric proteins used in methods of the present invention.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434FN436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, 1307A, E380A, 1253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AM mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V3081, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NO: 4 to SEQ ID NO: 50, or variants thereof are located between an extracellular domain as disclosed herein and an Fc domain as disclosed herein.

In embodiments, the chimeric proteins used in methods of the present invention may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 4 to SEQ ID NO: 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatenated oligomers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack an Fc domain linker, as disclosed herein.

In embodiments, the first and/or second joining linkers are independently selected from the amino acid sequences of SEQ ID NO: 4 to SEQ ID NO: 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDTLMISRTPEVTCVWDVSQEDPEVQFNWYVDGVEVHNAKTKPR |
| | EEQFNSTYRWSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVMHEALHNHYTQKSLSLSLGK |
| 2 | APEFLGGPSVFLFPPKPKDQLMISRTPEVICWVDVSQEDPEVQFNWYVDGVEVHNAKTKPR |
| | EEQFNSTYRVVSVLTTPHSDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVLHEALHNHYTQKSLSLSLGK |
| 3 | APEFLGGPSVFLFPPKPKDQLMISRTPEVICWVDVSQEDPEVQFNWYVDGVEVHNAKTKPR |
| | EEQFNSTYRWSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSRW |
| | QEGNVFSCSVLHEALHNHYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly) n where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

In embodiments, where a chimeric protein used in a method of the present invention comprises an extracellular domain (ECD) of a first transmembrane protein, one joining linker preceding an Fc domain, a second joining linker following the Fc domain, and an ECD of second transmembrane protein, the chimeric protein may comprise the following structure:
ECD-Joining Linker 1-Fc Domain-Joining Linker 2-ECD.

The combination of a first joining linker, an Fc Domain linker, and a second joining linker is referend to herein as a “modular linker”. In embodiments, a chimeric protein used in a method of the present invention comprises a modular linker as shown in Table 2:

TABLE 2
Illustrative modular linkers

| | | | Modular Linker = Joining |
|---|---|---|---|
| Joining Linker | | Joining | Linker 1 + Fc + Joining Linker |
| 1 | Fc | Linker 2 | 2 |
| SKYGPPCPSC | APEFLGGPSVFLFPPKPKDTL | IEGRMD | SKYGPPCPSCPAPEFLGGPSV |
| P | MISRTPEVTCWVDVSQEDPE | (SEQ ID NO: | FLFPPKPKDTLMISRTPEVTCV |
| (SEQ ID NO: 4) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 51) |
| SKYGPPCPSC | APEFLGGPSVFLFPPKPKDQL | IEGRMD | SKYGPPCPSCPAPEFLGGPSV |
| P | MISRTPEVTCWVDVSQEDPE | (SEQ ID NO: | FLFPPKPKDQLMISRTPEVTCV |
| (SEQ ID NO: 4) | VQFNWYVDGVEVHNAKTKPR | 7) | VVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 52) |
| SKYGPPCPSC | APEFLGGPSVFLFPPKPKDQL | IEGRMD | SKYGPPCPSCPAPEFLGGPSV |
| P | MISRTPEVTCWVDVSQEDPE | (SEQ ID NO: | FLFPPKPKDQLMISRTPEVTCV |
| (SEQ ID NO: 4) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 53) |
| SKYGPPCPPC | APEFLGGPSVFLFPPKPKDTL | IEGRMD | SKYGPPCPPCPAPEFLGGPSV |
| P | MISRTPEVTCWVDVSQEDPE | (SEQ ID NO: | FLFPPKPKDTLMISRTPEVTCV |
| (SEQ ID NO: 5) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 54) |
| SKYGPPCPPC | APEFLGGPSVFLFPPKPKDQL | IEGRMD | SKYGPPCPPCPAPEFLGGPSV |
| P | MISRTPEVTCWVDVSQEDPE | (SEQ ID NO: | FLFPPKPKDQLMISRTPEVTCV |
| (SEQ ID NO: 5) | VQFNWYVDGVEVHNAKTKPR | 7) | VVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 55) |
| SKYGPPCPPC | APEFLGGPSVFLFPPKPKDQL | IEGRMD | SKYGPPCPPCPAPEFLGGPSV |
| P | MISRTPEVTCWVDVSQEDPE | (SEQ ID NO: | FLFPPKPKDQLMISRTPEVTCV |
| (SEQ ID NO: 5) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 56) |

In embodiments, the chimeric proteins used in methods of the present invention may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 51 to SEQ ID NO: 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST | Flexible + soluble |
| (SEQ ID NO: 12) | |
| GGSG (SEQ ID NO: 13) | Flexible |
| GGSGGGSGGGSG (SEQ ID NO: 14) | Flexible |
| EAAAKEAAAKEAAAK | Rigid Alpha Helix |
| (SEQ ID NO: 15) | |
| EAAAREAAAREAAAREAAAR | Rigid Alpha Helix |
| (SEQ ID NO: 16) | |
| GGGGSGGGGSGGGGSAS | Flexible |
| (SEQ ID NO: 17) | |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP | Rigid |
| (SEQ ID NO: 23) | |

In embodiments, the linker may be functional. For example, without limitation, the linker may function to improve the folding and/or stability, improve the expression, improve the pharmacokinetics, and/or improve the bioactivity of the chimeric protein used in a method of the present invention. In another example, the linker may function to target the chimeric protein to a particular cell type or location.

In embodiments, a chimeric protein used in a method of the present invention comprises only one joining linkers.

In embodiments, a chimeric protein used in a method of the present invention lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or a portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly) n where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

Linkers described in one or more of WO2018/157162; WO2018/157165; WO2018/157164; WO2018/157163; and WO2017/059168 may be used, without limitation, in the present invention. The contents of each of which is incorporated herein by reference in its entirety.

In embodiments, a heterologous chimeric protein comprises a first domain comprising a portion of TIGIT, a second domain comprising a portion of OX40L, and a linker. In embodiments, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain, e.g., from an IgG1 or from IgG4, including human IgG1 or IgG4. In embodiments, the linker comprises an amino acid sequence that is at least 95% identical to the amino acid sequence of SEQ ID NO: 1, SEQ ID NO: 2, or SEQ ID NO: 3. Thus, in embodiments, when a heterologous chimeric protein used in a method of the present invention comprises the extracellular domain of TIGIT (or a variant thereof), a linker comprising a hinge-CH2-CH3 Fc domain, and the extracellular domain of OX40L (or a variant thereof), it may be referred to herein as “TIGIT-3-Fc-OX40L”.

In embodiments, a heterologous chimeric protein comprises a first domain comprising a portion of TIGIT, a second domain comprising a portion of LIGHT, and a linker. In embodiments, the linker is a polypeptide selected from a flexible amino acid sequence, an IgG hinge region, and an antibody sequence. In embodiments, the linker comprises at least one cysteine residue capable of forming a disulfide bond and/or comprises a hinge-CH2-CH3 Fc domain. In embodiments, the linker comprises a hinge-CH2-CH3 Fc domain, e.g., from an IgG1 or from IgG4, including human IgG1 or IgG4. In embodiments, the linker comprises an amino acid sequence that is at least 95% identical to the amino acid sequence of SEQ ID NO: 1, SEQ ID NO: 2, or SEQ ID NO: 3. Thus, in embodiments, when a heterologous chimeric protein used in a method of the present invention comprises the extracellular domain of TIGIT (or a variant thereof), a linker comprising a hinge-CH2-CH3 Fc domain, and the extracellular domain of LIGHT (or a variant thereof), it may be referred to herein as “TIGIT-3-Fc-LIGHT”.

Diseases, Methods of Treatment, and Mechanisms of Action

The methods comprise steps of administering to a subject in need thereof (either simultaneously or sequentially) an effective amount of at least one antibody directed to an immune checkpoint molecule; a stimulator of interferon genes (STING) agonist; and/or one or more chimeric proteins, in which each chimeric protein is capable of blocking immune inhibitory signals and/or stimulating immune activating signals. In embodiments, the chimeric protein comprises a first domain comprising a portion of TIGIT, a second domain comprising a portion of OX40L, and a linker. In embodiments, the chimeric protein comprises a first domain comprising a portion of TIGIT, a second domain comprising a portion of LIGHT, and a linker.

It is often desirable to disrupt, block, reduce, inhibit, and/or sequester the transmission of immune inhibitory signals and, simultaneously or contemporaneously, enhance, increase, and/or stimulate the transmission of an immune stimulatory signal to an anti-cancer immune cell, to boost an immune response, for instance to enhance a patient's anti-tumor immune response.

In embodiments, the antibodies directed to immune checkpoint molecules, STING agonists, and/or chimeric proteins used in methods of the present invention are capable of, or can be used in methods comprising, modulating the amplitude of an immune response, e.g., modulating the level of effector output.

In embodiments, e.g., when used for the treatment of cancer, the antibodies directed to immune checkpoint molecules; STING agonists; and/or chimeric proteins used in methods of the present invention alter the extent of immune stimulation as compared to immune inhibition to increase the amplitude of a T cell response, including, without limitation, stimulating increased levels of cytokine production, proliferation or target killing potential. In embodiments, the patient's T cells are activated and/or stimulated by the antibodies directed to immune checkpoint molecules, STING agonists, and/or chimeric proteins used in methods of the present invention, with the activated T cells being capable of dividing and/or secreting cytokines.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a colorectal cancer cell metastasizes to the liver, the secondary tumor, while present in the liver, is made up of colon cells or rectal cells, not of abnormal liver cells. The tumor in the liver may thus be a metastatic colorectal cancer, not liver cancer.

In embodiments, the antibodies directed to immune checkpoint molecules, STING agonists, and/or chimeric proteins used in methods of the present invention treat a subject that has a treatment-refractory cancer. In embodiments, the antibodies directed to immune checkpoint molecules, STING agonists, and/or chimeric proteins used in methods of the present invention treat a subject that is refractory to one or more immune-modulating agents. For example, in embodiments, the antibodies directed to immune checkpoint molecules, STING agonists, and/or chimeric proteins used in methods of the present invention treat a subject that presents no response to treatment, or even progress, after 12 weeks or so of treatment. For instance, in embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, in embodiments, the subject is refractory to an anti-CTLA-4 agent, e.g., ipilimumab (YERVOY)-refractory patients (e.g., melanoma patients). Accordingly, in embodiments the present invention provides methods of cancer treatment that rescue patients that are non-responsive to various therapies, including monotherapy of one or more immune-modulating agents.

In embodiments, the present invention provides antibodies directed to immune checkpoint molecules, STING agonists, and/or chimeric proteins which target a cell or tissue within the tumor microenvironment. In embodiments, the cell or tissue within the tumor microenvironment expresses one or more targets or binding partners of the antibodies directed to immune checkpoint molecules, STING agonists, and/or chimeric proteins used in methods of the present invention. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In embodiments, the antibodies directed to immune checkpoint molecules, STING agonists, and/or chimeric proteins used in methods of the present invention targets a cancer cell. In embodiments, the cancer cell expresses one or more of targets or binding partners of the antibodies directed to immune checkpoint molecules, STING agonists, and/or chimeric proteins used in methods of the present invention.

The activation of regulatory T cells is critically influenced by costimulatory and co-inhibitory signals. Two major families of costimulatory molecules include the B7 and the tumor necrosis factor (TNF) families. These molecules bind to receptors on T cells belonging to the CD28 or TNF receptor families, respectively. Many well-defined co-inhibitors and their receptors belong to the B7 and CD28 families.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity, or phagocytic activity of leukocytes. Specific examples include direct stimulation of TNF superfamily receptors such as OX40, LTbR, 4-1BB, or TNFRSF25 using either receptor agonist antibodies or using a chimeric protein comprising the ligands for such receptors (OX40L, LIGHT, 4-1BBL, and TL1A, respectively). Stimulation from any one of these receptors may directly stimulate the proliferation and cytokine production of individual T cell subsets. Another example includes direct stimulation of an immune inhibitory cell with through a receptor that inhibits the activity of such an immune suppressor cell.

In embodiments, the antibodies directed to immune checkpoint molecules, STING agonists, and/or chimeric proteins are capable of, or find use in methods involving, enhancing, restoring, promoting and/or stimulating immune modulation. In embodiments, the antibodies directed to immune checkpoint molecules, STING agonists, and/or chimeric proteins used in methods of the present invention described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In embodiments, the antibodies directed to immune checkpoint molecules, STING agonists, and/or chimeric proteins used in methods of the present invention enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: pro-inflammatory cytokine production or T cell migration or T cell tumor infiltration.

In embodiments, the antibodies directed to immune checkpoint molecules, STING agonists, and/or chimeric proteins used in methods of the present invention are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the antibodies directed to immune checkpoint molecules, STING agonists, and/or chimeric proteins used in methods of the present invention enhance recognition of tumor antigens by CD8+ T cells, particularly those T cells that have infiltrated into the tumor microenvironment. In embodiments, the antibodies directed to immune checkpoint molecules, STING agonists, and/or chimeric proteins used in methods of the present invention induce CD19 expression and/or increases the number of CD19 positive cells (e.g., CD19 positive B cells). In embodiments, the antibodies directed to immune checkpoint molecules, STING agonists, and/or chimeric proteins used in methods of the present invention induce IL-15Ra expression and/or increases the number of IL-15Ra positive cells (e.g., IL-15Ra positive dendritic cells).

In embodiments, the antibodies directed to immune checkpoint molecules, STING agonists, and/or chimeric proteins used in methods of the present invention are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs)), and particularly within the tumor and/or tumor microenvironment (TME). In embodiments, the present therapies may alter the ratio of M1 versus M2 macrophages in the tumor site and/or TME to favor M1 macrophages.

In embodiments, the antibodies directed to immune checkpoint molecules, STING agonists, and/or chimeric proteins used in methods of the present invention are able to increase the serum levels of various cytokines or chemokines including, but not limited to, one or more of IFNγ, TNFα, IL-2, IL-4, IL-5, IL-6, IL-7, IL-9, IL-10, IL-13, IL-15, IL-17A, IL-17F, IL-22, CCL2, CCL3, CCL4, CXCL8, CXCL9, CXCL10, CXCL11 and CXCL12. In embodiments, the antibodies directed to immune checkpoint molecules, STING agonists, and/or chimeric proteins used in methods of the present invention are capable of enhancing IL-2, IL-4, IL-5, IL-10, IL-13, IL-17A, IL-22, TNFα or IFNγ in the serum of a treated subject. In embodiments, administration of the antibodies directed to immune checkpoint molecules, STING agonists, and/or chimeric proteins used in methods of the present invention is capable of enhancing TNFα secretion. In a specific embodiment, administration of the antibodies directed to immune checkpoint molecules, STING agonists, and/or chimeric proteins used in methods of the present invention is capable of enhancing superantigen mediated TNFα secretion by leukocytes. Detection of such a cytokine response may provide a method to determine the optimal dosing regimen for the indicated antibodies directed to immune checkpoint molecules, STING agonists, and/or chimeric proteins used in methods of the present invention.

The antibodies directed to immune checkpoint molecules, STING agonists, and/or chimeric proteins used in methods of the present invention are capable of increasing or preventing a decrease in a sub-population of CD4+ and/or CD8+ T cells.

The antibodies directed to immune checkpoint molecules, STING agonists, and/or chimeric proteins used in methods of the present invention are capable of enhancing tumor killing activity by T cells.

In embodiments, the antibodies directed to immune checkpoint molecules, STING agonists, and/or chimeric proteins used in methods of the present invention inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections, inflammatory diseases, and cancer. This dysfunction is defined by poor proliferative and/or effector functions, sustained expression of inhibitory receptors and a transcriptional state distinct from that of functional effector or memory T cells. Exhaustion prevents optimal control of infection and tumors. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refer to receptors expressed on immune cells that prevent or inhibit uncontrolled immune responses. In contrast, an anti-tumor CD8+ and/or CD4+ T cell refers to T cells that can mount an immune response to a tumor.

In embodiments, the antibodies directed to immune checkpoint molecules, STING agonists, and/or chimeric proteins used in methods of the present invention are capable of, and can be used in methods comprising, increasing a ratio of effector T cells to regulatory T cells. Illustrative effector T cells include ICOS+ effector T cells; cytotoxic T cells (e.g., αβ TCR, CD3+, CD8+, CD45RO⁺); CD4+ effector T cells (e.g., αβ TCR, CD3⁺, CD4⁺, CCR7⁺, CD62Lhi, IL⁻7R/CD127⁺); CD8⁺ effector T cells (e.g., αβ TCR, CD3⁺, CD8⁺, CCR7⁺, CD62Lhi, IL⁻7R/CD127⁺); effector memory T cells (e.g., CD62Llow, CD44⁺, TCR, CD3⁺, IL⁻7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27+CD62L⁻) and late effector memory T cells (CD27⁻ CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4+, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(⁻) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-β, TNF-α, Galectin-1, IFN-γ and/or MCP1.

In embodiments, the antibodies directed to immune checkpoint molecules, STING agonists, and/or chimeric proteins used in methods of the present invention cause an increase in effector T cells (e.g., CD4+CD25− T cells).

In embodiments, the antibodies directed to immune checkpoint molecules, STING agonists, and/or chimeric proteins used in methods of the present invention cause a decrease in regulatory T cells (e.g., CD4+CD25+ T cells).

In embodiments, the antibodies directed to immune checkpoint molecules, STING agonists, and/or chimeric proteins used in methods of the present invention generate a memory response which may, e.g., be capable of preventing relapse or protecting the animal from a recurrence and/or preventing, or reducing the likelihood of, metastasis. Thus, an animal treated with the antibodies directed to immune checkpoint molecules, STING agonists, and/or chimeric proteins used in methods of the present invention is later able to attack tumor cells and/or prevent development of tumors when re-challenged after an initial treatment with the antibodies directed to immune checkpoint molecules, STING agonists, and/or chimeric proteins used in methods of the present invention. Accordingly, the antibodies directed to immune checkpoint molecules, STING agonists, and/or chimeric proteins used in methods of the present invention stimulate both active tumor destruction and also immune recognition of tumor antigens, which are essential in programming a memory response capable of preventing relapse.

In embodiments, the antibodies directed to immune checkpoint molecules, STING agonists, and/or chimeric proteins used in methods of the present invention are capable of causing activation of antigen presenting cells. In embodiments, the antibodies directed to immune checkpoint molecules, STING agonists, and/or chimeric proteins used in methods of the present invention are capable enhancing the ability of antigen presenting cells to present antigen.

In embodiments, the antibodies directed to immune checkpoint molecules, STING agonists, and/or chimeric proteins used in methods of the present invention are capable of, and can be used in methods comprising, transiently stimulating effector T cells for longer than about 12 hours, about 24 hours, about 48 hours, about 72 hours or about 96 hours or about 1 week or about 2 weeks. In embodiments, the transient stimulation of effector T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

The chimeric proteins used in methods of the present invention unexpectedly provide binding of the extracellular domain components to their respective binding partners with slow off rates (Kd or K_{off}). In embodiments, this provides an unexpectedly long interaction of the receptor to ligand and vice versa. Such an effect allows for a longer positive signal effect, e.g., increase in or activation of immune stimulatory signals. For example, the chimeric proteins used in methods of the present invention, e.g., via the long off rate binding allows sufficient signal transmission to provide immune cell proliferation, allow for anti-tumor attack, allows sufficient signal transmission to provide release of stimulatory signals, e.g., cytokines.

The chimeric proteins used in methods of the present invention are capable of forming a stable synapse between cells. The stable synapse of cells promoted by the chimeric proteins (e.g., between cells bearing negative signals) provides spatial orientation to favor tumor reduction—such as positioning the T cells to attack tumor cells and/or sterically preventing the tumor cell from delivering negative signals, including negative signals beyond those masked by the chimeric proteins. In embodiments, this provides longer on-target (e.g., intra-tumoral) half-life (t112) as compared to serum t112 of the chimeric proteins. Such properties could have the combined advantage of reducing off-target toxicities associated with systemic distribution of the chimeric proteins.

In embodiments, the antibodies directed to immune checkpoint molecules, STING agonists, and/or chimeric proteins used in methods of the present invention are capable of providing a sustained immunomodulatory effect.

The antibodies directed to immune checkpoint molecules, STING agonists, and/or chimeric proteins used in methods of the present invention provide synergistic therapeutic effects (e.g., anti-tumor effects) as it allows for improved site-specific interplay of two immunotherapy agents. In embodiments, the antibodies directed to immune checkpoint molecules, STING agonists, and/or chimeric proteins used in methods of the present invention provide the potential for reducing off-site and/or systemic toxicity.

In embodiments, the chimeric proteins used in methods of the present invention exhibit enhanced safety profiles. In embodiment, the chimeric proteins used in methods of the present invention exhibit reduced toxicity profiles. For example, administration of the chimeric proteins used in methods of the present invention may result in reduced side effects such as one or more of diarrhea, inflammation (e.g., of the gut), or weight loss, which occur following administration of antibodies directed to the ligand(s)/receptor(s) targeted by the extracellular domains of the chimeric proteins used in methods of the present invention used in methods of the present invention. In embodiments, the chimeric proteins used in methods of the present invention provides improved safety, as compared to antibodies directed to the ligand(s)/receptor(s) targeted by the extracellular domains of the chimeric proteins used in methods of the present invention used in methods of the present invention, yet, without sacrificing efficacy.

In embodiments, the chimeric proteins used in methods of the present invention provide reduced side-effects, e.g., GI complications, relative to current immunotherapies, e.g., antibodies directed to ligand(s)/receptor(s) targeted by the extracellular domains of the chimeric proteins used in methods of the present invention used in methods of the present invention. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

Methods of Treatment

In various aspects, the present invention provides compositions and methods that are useful for cancer immunotherapy. For instance, the present invention, in part, relates to methods for treating cancer comprising steps of administering to a subject in need thereof (either simultaneously or sequentially) an effective amount of at least one antibody directed to an immune checkpoint molecule; a stimulator of interferon genes (STING) agonist; and/or one or more chimeric proteins. In embodiments, the chimeric protein comprises a first domain comprising a portion of TIGIT, a second domain comprising a portion of OX40L, and a linker.

An aspect of the present invention relates to a method for treating a cancer in a subject in need thereof comprising: providing the subject a first pharmaceutical composition comprising an antibody that is capable of binding cytotoxic T lymphocyte-associated antigen 4 (CTLA-4) and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, dose of the first pharmaceutical composition is less than the dose of the first pharmaceutical composition provided to a subject who has not undergone or is not undergoing treatment with the second pharmaceutical composition.

In embodiments, the dose of the second pharmaceutical composition provided is less than the dose of the second pharmaceutical composition provided to a subject who has not undergone or is not undergoing treatment with the first pharmaceutical composition.

In embodiments, the first pharmaceutical composition and the second pharmaceutical composition are provided simultaneously. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition

In embodiments, the first pharmaceutical composition is provided after the second pharmaceutical composition is provided. In embodiments, dose of the first pharmaceutical composition is less than the dose of the first pharmaceutical composition provided to a subject who has not undergone or is not undergoing treatment with the second pharmaceutical composition.

In embodiments, the first pharmaceutical composition is provided before the second pharmaceutical composition is provided. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

Another aspect of the present invention relates to a method for treating a cancer in a subject who has undergone or is undergoing treatment with an antibody that is capable of binding cytotoxic T lymphocyte-associated antigen 4 (CTLA-4). The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

In embodiments, the subject has an increased chance of survival, a gain in weight, and/or a reduction in tumor size or cancer prevalence when compared to the subject who has not undergone or is not undergoing treatment with an antibody that is capable of binding PD-1 or binding a PD-1 ligand.

Yet another aspect of the present invention provides a method for treating a cancer in a subject comprising providing the subject a pharmaceutical composition comprising an antibody that is capable of binding cytotoxic T lymphocyte-associated antigen 4 (CTLA-4). Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with the heterologous chimeric protein.

In embodiments, the subject has a cancer that is poorly responsive or is refractory to treatment comprising the antibody that is capable of binding PD-1 or binding a PD-1 ligand.

In embodiments, the first domain comprises substantially all of the extracellular domain of TIGIT and/or the second domain comprises substantially all of the extracellular domain of OX40L.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG1 or IgG4, e.g., human IgG1 or human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the subject has a cancer that is poorly responsive or is refractory to treatment comprising an antibody that is capable of binding PD-1 or binding a PD-1 ligand.

In embodiments, the cancer is poorly responsive or is non-responsive to treatment with an antibody that is capable of binding PD-1 or binding a PD-1 ligand after 12 weeks or so of such treatment.

In embodiments, the antibody that is capable of binding CTLA-4. Illustrative antibodies capable of binding CTLA-4 include YERVOY (ipilimumab), 9D9, tremelimumab (formerly ticilimumab, CP-675,206; MedImmune), AGEN1884, and RG2077.

In embodiments, the cancer is a cancer suitable for treatment with an antibody that is capable of binding CTLA-4. Illustrative antibodies capable of binding CTLA-4 include YERVOY (ipilimumab), 9D9, tremelimumab (formerly ticilimumab, CP-675,206; MedImmune), AGEN1884, and RG2077. Such an antibody contributes to cancer treatment, in part, by inhibiting the interaction of CTLA-4 with one or more of its ligands.

An aspect of the present invention relates to a method for treating a cancer in a subject in need thereof comprising: providing the subject a first pharmaceutical composition comprising an antibody that is capable of binding programmed cell death protein 1 (PD-1) or binding a PD-1 ligand and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, dose of the first pharmaceutical composition is less than the dose of the first pharmaceutical composition provided to a subject who has not undergone or is not undergoing treatment with the second pharmaceutical composition.

In embodiments, the dose of the second pharmaceutical composition provided is less than the dose of the second pharmaceutical composition provided to a subject who has not undergone or is not undergoing treatment with the first pharmaceutical composition.

In embodiments, the first pharmaceutical composition and the second pharmaceutical composition are provided simultaneously. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition

In embodiments, the first pharmaceutical composition is provided after the second pharmaceutical composition is provided. In embodiments, dose of the first pharmaceutical composition is less than the dose of the first pharmaceutical composition provided to a subject who has not undergone or is not undergoing treatment with the second pharmaceutical composition.

In embodiments, the first pharmaceutical composition is provided before the second pharmaceutical composition is provided. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

Another aspect of the present invention relates to a method for treating a cancer in a subject who has undergone or is undergoing treatment with an antibody that is capable of binding programmed cell death protein 1 (PD-1) or binding a PD-1 ligand. The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

In embodiments, the subject has an increased chance of survival, without gastrointestinal inflammation and weight loss, and/or a reduction in tumor size or cancer prevalence when compared to the subject who has not undergone or is not undergoing treatment with an antibody that is capable of binding PD-1 or binding a PD-1 ligand.

Yet another aspect of the present invention provides a method for treating a cancer in a subject comprising providing the subject a pharmaceutical composition comprising an antibody that is capable of binding programmed cell death protein 1 (PD-1) or binding a PD-1 ligand. Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with the heterologous chimeric protein.

In embodiments, the subject has a cancer that is poorly responsive or is refractory to treatment comprising the antibody that is capable of binding PD-1 or binding a PD-1 ligand.

In embodiments, the first domain comprises substantially all of the extracellular domain of TIGIT and/or the second domain comprises substantially all of the extracellular domain of OX40L.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG1 or IgG4, e.g., human IgG1 or human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the subject has a cancer that is poorly responsive or is refractory to treatment comprising an antibody that is capable of binding PD-1 or binding a PD-1 ligand.

In embodiments, the cancer is poorly responsive or is non-responsive to treatment with an antibody that is capable of binding PD-1 or binding a PD-1 ligand after 12 weeks or so of such treatment.

In embodiments, the antibody is capable of binding PD-1 or a PD-1 ligand. Illustrative antibodies capable of binding PD-1 or a PD-1 ligand include nivolumab (ONO 4538, BMS 936558, MDX1106, OPDIVO (Bristol Myers Squibb)), pembrolizumab (KEYTRUDA/MK 3475, Merck), pidilizumab (CT 011, Cure Tech), RMP1-14, AGEN2034 (Agenus), and cemiplimab ((REGN-2810). Such an antibody is capable of inhibiting the interaction of PD-1 with one or more of its ligands.

In embodiments, the cancer is a cancer suitable for treatment with an antibody that is capable of binding PD-1 or a PD-1 ligand. Illustrative antibodies capable of binding PD-1 or a PD-1 ligand include nivolumab (ONO 4538, BMS 936558, MDX1106, OPDIVO (Bristol Myers Squibb)), pembrolizumab (KEYTRUDA/MK 3475, Merck), pidilizumab (CT 011, Cure Tech), RMP1-14, AGEN2034 (Agenus), and cemiplimab ((REGN-2810). Such an antibody contributes to cancer treatment, in part, by inhibiting the interaction of PD-1 with one or more of its ligands.

An aspect of the present invention relates to a method for treating a cancer in a subject in need thereof comprising: providing the subject a first pharmaceutical composition comprising a stimulator of interferon genes (STING) agonist and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, dose of the first pharmaceutical composition is less than the dose of the first pharmaceutical composition provided to a subject who has not undergone or is not undergoing treatment with the second pharmaceutical composition.

In embodiments, the dose of the second pharmaceutical composition provided is less than the dose of the second pharmaceutical composition provided to a subject who has not undergone or is not undergoing treatment with the first pharmaceutical composition.

In embodiments, the first pharmaceutical composition and the second pharmaceutical composition are provided simultaneously. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition

In embodiments, the first pharmaceutical composition is provided after the second pharmaceutical composition is provided. In embodiments, dose of the first pharmaceutical composition is less than the dose of the first pharmaceutical composition provided to a subject who has not undergone or is not undergoing treatment with the second pharmaceutical composition.

In embodiments, the first pharmaceutical composition is provided before the second pharmaceutical composition is provided. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

Another aspect of the present invention relates to a method for treating a cancer in a subject who has undergone or is undergoing treatment with a stimulator of interferon genes (STING) agonist. The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

In embodiments, the subject has an increased chance of survival, without gastrointestinal inflammation and weight loss, and/or a reduction in tumor size or cancer prevalence when compared to the subject who has not undergone or is not undergoing treatment with an antibody that is capable of binding PD-1 or binding a PD-1 ligand.

Yet another aspect of the present invention provides a method for treating a cancer in a subject comprising providing the subject a pharmaceutical composition comprising a stimulator of interferon genes (STING) agonist. Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with the heterologous chimeric protein.

In embodiments, the subject has a cancer that is poorly responsive or is refractory to treatment comprising the antibody that is capable of binding PD-1 or binding a PD-1 ligand.

In embodiments, the first domain comprises substantially all of the extracellular domain of TIGIT and/or the second domain comprises substantially all of the extracellular domain of OX40L.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG1 or IgG4, e.g., human IgG1 or human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the subject has a cancer that is poorly responsive or is refractory to treatment comprising an antibody that is capable of binding PD-1 or binding a PD-1 ligand.

In embodiments, the cancer is poorly responsive or is non-responsive to treatment with an antibody that is capable of binding PD-1 or binding a PD-1 ligand after 12 weeks or so of such treatment.

In embodiments, the STING Agonist is selected from the group consisting of 5,6-dimethylxanthenone-4-acetic acid (DMXAA), MIW815(ADU-5100), CRD5500, MK-1454, SB11285, IMSA101, and any STING agonist described in US20140341976, US20180028553, US20180230178, U.S. Pat. No. 9,549,944, WO2015185565, WO2016120305, WO2017044622, WO2017027645, WO2017027646, WO2017093933, WO2017106740, WO2017123657, WO2017123669, WO2017161349, WO2017175147, WO2017175156, WO2017176812, WO2018009466, WO2018045204, WO2018060323, WO2018098203, WO2018100558, WO2018138684, WO2018138685, WO2018152450, WO2018152453, WO2018172206, WO2018198084, WO2018234805, WO2018234807, WO2018234808, WO2019023459, WO2019046496, WO2019046498, WO2019046500, WO2019074887, WO2019079261, WO2019118839, WO2019125974, or WO2019160884, he contents of which are incorporated herein by reference in their entireties. In embodiments, the STING agonist is selected from the group consisting of 5,6-dimethylxanthenone-4-acetic acid (DMXAA), MIW815(ADU-5100), CRD5500, MK-1454, SB11285, IMSA101.

In embodiments, the cancer is a cancer suitable for treatment with a STING agonist. Illustrative STING agonists include 5,6-dimethylxanthenone-4-acetic acid (DMXAA), MIW815(ADU-S100), CRD5500, MK-1454, SB11285, IMSA101, and any STING agonist described in US20140341976, US20180028553, US20180230178, U.S. Pat. No. 9,549,944, WO2015185565, WO2016120305, WO2017044622, WO2017027645, WO2017027646, WO2017093933, WO2017106740, WO2017123657, WO2017123669, WO2017161349, WO2017175147, WO2017175156, WO2017176812, WO2018009466, WO2018045204, WO2018060323, WO2018098203, WO2018100558, WO2018138684, WO2018138685, WO2018152450, WO2018152453, WO2018172206, WO2018198084, WO2018234805, WO2018234807, WO2018234808, WO2019023459, WO2019046496, WO2019046498, WO2019046500, WO2019074887, WO2019079261, WO2019118839, WO2019125974, or WO2019160884, he contents of which are incorporated herein by reference in their entireties, the contents of which are incorporated herein by reference in their entireties. Such STING agonists contribute to cancer treatment, in part, by promoting immune activation and stimulating immune cells to attack the cancer.

In aspects and embodiments of the present invention, a patient in need of a cancer treatment comprising an antibody directed to immune checkpoint molecules; STING agonist; and/or chimeric protein used in methods of the present invention, as disclosed herein, is or is predicted to be poorly responsive or is non-responsive to an immunotherapy, e.g., an anti-cancer immunotherapy, as disclosed herein. Moreover, in embodiments, a patient in need of an anti-cancer agent, as disclosed herein, is or may is predicted to be poorly responsive or non-responsive to an immune checkpoint immunotherapy. The immune checkpoint molecule may be selected from PD-1, PD-L1, PD-L2, ICOS, ICOSL, and CTLA-4.

Examples of cancers suitable for treatment according to the present invention include Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, and urothelial carcinoma.

An aspect of the present invention relates to a method for treating a cancer in a subject in need thereof comprising: providing the subject a first pharmaceutical composition comprising an antibody that is capable of binding programmed cell death protein 1 (PD-1) or binding a PD-1 ligand and providing the subject a second pharmaceutical composition comprising a heterologous chimeric protein. Here, the heterologous chimeric protein comprises: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of LIGHT, wherein the portion is capable of binding a LIGHT receptor, and (c) a linker linking the first domain and the second domain.

In embodiments, dose of the first pharmaceutical composition is less than the dose of the first pharmaceutical composition provided to a subject who has not undergone or is not undergoing treatment with the second pharmaceutical composition.

In embodiments, the dose of the second pharmaceutical composition provided is less than the dose of the second pharmaceutical composition provided to a subject who has not undergone or is not undergoing treatment with the first pharmaceutical composition.

In embodiments, the first pharmaceutical composition and the second pharmaceutical composition are provided simultaneously. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition

In embodiments, the first pharmaceutical composition is provided after the second pharmaceutical composition is provided. In embodiments, dose of the first pharmaceutical composition is less than the dose of the first pharmaceutical composition provided to a subject who has not undergone or is not undergoing treatment with the second pharmaceutical composition.

In embodiments, the first pharmaceutical composition is provided before the second pharmaceutical composition is provided. In embodiments, dose of the first pharmaceutical composition is less than the dose of the first pharmaceutical composition provided to a subject who has not undergone or is not undergoing treatment with the second pharmaceutical composition. In embodiments, the dose of the second pharmaceutical composition provided is less than the dose of the second pharmaceutical composition provided to a subject who has not undergone or is not undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition.

In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

Another aspect of the present invention relates to a method for treating a cancer in a subject who has undergone or is undergoing treatment with an antibody that is capable of binding programmed cell death protein 1 (PD-1) or binding a PD-1 ligand. The method comprises providing the subject a pharmaceutical composition comprising a heterologous chimeric comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of LIGHT, wherein the portion is capable of binding a LIGHT receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with an antibody that is capable of binding PD 1 or binding a PD 1 ligand.

In embodiments, the subject has an increased chance of survival, a gain in weight, and/or a reduction in tumor size or cancer prevalence when compared to the subject who has not undergone or is not undergoing treatment with an antibody that is capable of binding PD 1 or binding a PD 1 ligand.

Yet another aspect of the present invention provides a method for treating a cancer in a subject comprising providing the subject a pharmaceutical composition comprising an antibody that is capable of binding programmed cell death protein 1 (PD-1) or binding a PD-1 ligand. Here, the subject has undergone or is undergoing treatment with a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of LIGHT, wherein the portion is capable of binding a LIGHT receptor, and (c) a linker linking the first domain and the second domain.

In embodiments, the dose of the pharmaceutical composition provided to the subject is less than the dose of the pharmaceutical composition that is provided to a subject who has not undergone or is not undergoing treatment with the heterologous chimeric protein.

In embodiments, the subject has a cancer that is poorly responsive or is refractory to treatment comprising the antibody that is capable of binding PD 1 or binding a PD 1 ligand.

In embodiments, the first domain comprises substantially all of the extracellular domain of TIGIT and/or the second domain comprises substantially all of the extracellular domain of LIGHT.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain. In embodiments, the linker comprises a hinge-CH2-CH3 Fc domain derived from IgG1 or IgG4, e.g., human IgG1 or human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the subject has a cancer that is poorly responsive or is refractory to treatment comprising an antibody that is capable of binding PD 1 or binding a PD 1 ligand.

In embodiments, the cancer is poorly responsive or is non-responsive to treatment with an antibody that is capable of binding PD 1 or binding a PD 1 ligand after 12 weeks or so of such treatment.

In embodiments, the antibody is capable of binding PD-1 or a PD-1 ligand. Illustrative antibodies capable of binding PD-1 or a PD-1 ligand include nivolumab (ONO 4538, BMS 936558, MDX1106, OPDIVO (Bristol Myers Squibb)), pembrolizumab (KEYTRUDA/MK 3475, Merck), and cemiplimab ((REGN-2810). Such an antibody is capable of inhibiting the interaction of PD-1 with one or more of its ligands.

In embodiments, the cancer is a cancer suitable for treatment with an antibody that is capable of binding PD-1 or a PD-1 ligand. Illustrative antibodies capable of binding PD-1 or a PD-1 ligand include nivolumab (ONO 4538, BMS 936558, MDX1106, OPDIVO (Bristol Myers Squibb)), pembrolizumab (KEYTRUDA/MK 3475, Merck), and cemiplimab ((REGN-2810). Such an antibody contributes to cancer treatment, in part, by inhibiting the interaction of PD-1 with one or more of its ligands.

Pharmaceutical Compositions

The methods of the present invention include administering pharmaceutical compositions comprising a therapeutically effective amount of an antibody directed to an immune checkpoint molecule and/or a STING agonist and a chimeric protein used in methods of the present invention, as disclosed herein.

The antibodies directed to immune checkpoint molecules; STING agonists; and/or chimeric proteins used in methods of the present invention disclosed herein can possess a sufficiently basic functional group, which can react with an inorganic or organic acid, or a carboxyl group, which can react with an inorganic or organic base, to form a pharmaceutically acceptable salt. A pharmaceutically-acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, any antibody directed to immune checkpoint molecules; STING agonist; and/or chimeric protein used in methods of the present invention disclosed herein can be administered to a subject as a component of a composition, e.g., pharmaceutical composition, which comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the compositions, e.g., pharmaceutical compositions, disclosed herein are resuspended in a saline buffer (including, without limitation TBS, PBS, and the like).

In embodiments, the antibodies directed to immune checkpoint molecules; STING agonists; and/or chimeric proteins used in methods of the present invention may by conjugated and/or fused with another agent to extend half-life or otherwise improve pharmacodynamic and pharmacokinetic properties. In embodiments, the antibodies directed to immune checkpoint molecules; STING agonists; and/or chimeric proteins used in methods of the present invention may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

The present invention includes the antibodies directed to immune checkpoint molecules; STING agonists; and/or chimeric proteins used in methods of the present invention in various formulations of pharmaceutical composition. Any antibody directed to immune checkpoint molecules; STING agonist; and/or chimeric protein used in methods of the present invention disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the antibodies directed to immune checkpoint molecules; STING agonists; and/or chimeric proteins used in methods of the present invention can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The pharmaceutical compositions comprising the antibodies directed to immune checkpoint molecules; STING agonists; and/or chimeric proteins used in methods of the present invention of the present invention may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any antibody directed to immune checkpoint molecules; STING agonist; and/or chimeric protein used in methods of the present invention disclosed herein is formulated in accordance with routine procedures as a pharmaceutical composition adapted for a mode of administration disclosed herein.

Administration, Dosing, and Treatment Regimens

As examples, administration results in the release of antibodies directed to immune checkpoint molecules; STING agonists; and/or chimeric proteins used in methods of the present invention disclosed herein into the bloodstream, or alternatively, the antibodies directed to immune checkpoint molecules; STING agonists; and/or chimeric proteins used in methods of the present invention is administered directly to the site of active disease.

Any antibody directed to immune checkpoint molecules; STING agonists; and/or chimeric proteins used in methods of the present invention can also be administered by any convenient route, for example, by intravenous infusion or bolus injection. Administration can be systemic or can be local (e.g., intra-tumoral injection). Various delivery systems are known, e.g., encapsulation in liposomes, microparticles, microcapsules, capsules, etc., and can be used to administer agents.

In specific embodiments, it may be desirable to administer locally to the area in need of treatment. In embodiments, for instance in the treatment of cancer, the antibodies directed to immune checkpoint molecules; STING agonists; and/or chimeric proteins used in methods of the present invention are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the antibodies directed to immune checkpoint molecules; STING agonists; and/or chimeric proteins used in methods of the present invention are administered intratumorally.

In embodiments, the antibodies directed to immune checkpoint molecules; STING agonists; and/or chimeric proteins used in methods of the present invention allows for a dual effect that provides less side effects than are seen in conventional immunotherapy (e.g., treatments with one or more of OPDIVO, KEYTRUDA, YERVOY, and TECENTRIQ). For example, the antibodies directed to immune checkpoint molecules; STING agonists; and/or chimeric proteins used in methods of the present invention reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any antibody directed to immune checkpoint molecules; STING agonist; and/or chimeric protein used in methods of the present invention disclosed herein as well as the dosing schedule can depend on various parameters, including, but not limited to, the disease being treated, the severity of the condition, whether the condition is to be treated or prevented, the age, weight, sex, medical condition, and health of the subject to be treated, the renal or hepatic function of the subject, the specific compound of the invention employed, the route of administration, and the administering physician's discretion. Additionally, pharmacogenomic (the effect of genotype on the pharmacokinetic, pharmacodynamic or efficacy profile of a therapeutic) information about a particular subject may affect dosage and does schedule used. Furthermore, the exact individual dosages and dosing schedules can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A antibody directed to immune checkpoint molecules; STING agonist; and/or chimeric protein used in methods of the present invention disclosed herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Furthermore, any antibody directed to immune checkpoint molecules; STING agonist; and/or chimeric protein used in methods of the present invention disclosed herein can be administered continuously rather than intermittently throughout the dosage regimen.

Fusion Proteins, Nucleic Acids, and Cells

A chimeric protein used in a method of the present invention may be a recombinant fusion protein, e.g., a single polypeptide having the extracellular domains disclosed herein. For example, in embodiments, the chimeric protein is translated as a single unit in a prokaryotic cell, a eukaryotic cell, or a cell-free expression system.

In embodiments, a chimeric protein is recombinant protein comprising multiple polypeptides, e.g., multiple extracellular domains disclosed herein, that are combined (via covalent or non-covalent bonding) to yield a single unit, e.g., in vitro (e.g., with one or more synthetic linkers disclosed herein).

In embodiments, a chimeric protein is chemically synthesized as one polypeptide or each domain may be chemically synthesized separately and then combined. In embodiments, a portion of the chimeric protein is translated and a portion is chemically synthesized.

Constructs could be produced by cloning of the nucleic acids encoding the three fragments (the extracellular domain of a Type I transmembrane protein, followed by a linker sequence, followed by the extracellular domain of a Type II transmembrane protein) into a vector (plasmid, viral or other) wherein the amino terminus of the complete sequence corresponded to the ‘left’ side of the molecule containing the extracellular domain of the Type I transmembrane protein and the carboxy terminus of the complete sequence corresponded to the ‘right’ side of the molecule containing the extracellular domain of Type II transmembrane protein. In embodiments of chimeric proteins having one of the other configurations, as described elsewhere herein, a construct would comprise three nucleic acids such that the translated chimeric protein produced would have the desired configuration, e.g., a dual inward-facing chimeric protein. Accordingly, in embodiments, the chimeric proteins used in methods of the present invention are engineered as such.

A chimeric protein used in a method of the present invention may be encoded by a nucleic acid cloned into an expression vector. In embodiments, the expression vector comprises DNA or RNA. In embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the Agt vector series such as Agt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors comprise a nucleic acid encoding the chimeric proteins, or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

In embodiments, a chimeric protein used in a method of the present invention is producible in a mammalian host cell as a secretable and fully functional single polypeptide chain.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In embodiments, the cell is a tumor cell. In another embodiment, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present invention contemplates the use of inducible promoters capable of effecting high level of expression transiently in response to a cue. For example, when in the proximity of a tumor cell, a cell transformed with an expression vector for the chimeric protein comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. In other examples, the chimeric protein is expressed by a chimeric antigen receptor containing cell or an in vitro expanded tumor infiltrating lymphocyte, under the control of a promoter which is sensitive to antigen recognition by the cell, and leads to local secretion of the chimeric protein in response to tumor antigen recognition. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In embodiments, the expression vectors for the expression of the chimeric proteins are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the invention provides methods of transducing a human cell in vivo, comprising contacting a solid tumor in vivo with a viral vector of the invention.

Expression vectors can be introduced into host cells for producing the chimeric proteins used in methods of the present invention. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins disclosed herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC#2 and SCLC#7.

Host cells can be obtained from normal or affected subjects, including healthy humans, cancer patients, and patients with an infectious disease, private laboratory deposits, public culture collections such as the American Type Culture Collection (ATCC), or from commercial suppliers.

Cells that can be used for production of the chimeric proteins used in methods of the present invention in vitro, ex vivo, and/or in vivo include, without limitation, epithelial cells, endothelial cells, keratinocytes, fibroblasts, muscle cells, hepatocytes; blood cells such as T lymphocytes, chimeric antigen receptor expressing T cells, tumor infiltrating lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, and fetal liver. The choice of cell type depends on the type of tumor or infectious disease being treated or prevented, and can be determined by one of skill in the art.

Production and purification of Fc-containing macromolecules (such as monoclonal antibodies) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In embodiments, production of the chimeric proteins contemplated herein may have unique characteristics as compared to traditional Fc containing macromolecules. In certain examples, the chimeric proteins may be purified using specific chromatography resins, or using chromatography methods that do not depend upon Protein A capture. In embodiments, the chimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The chimeric proteins obtained herein may be additionally ‘polished’ using methods that are specified in the art. In embodiments, the chimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In embodiments, the chimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use. In a specific embodiment, the non-human animal is a household pet. In another specific embodiment, the non-human animal is a livestock animal.

In embodiments, the subject has a cancer that is poorly responsive or is refractory to treatment comprising an antibody that is capable of binding PD-1 or binding a PD-1 ligand. In embodiments, the subject has a cancer that is poorly responsive or is non-responsive to treatment with an antibody that is capable of binding PD-1 or binding a PD-1 ligand after 12 weeks or so of such treatment.

Kits and Medicaments

Aspects of the present invention provide kits that can simplify the administration of the pharmaceutical compositions and/or chimeric proteins disclosed herein.

An illustrative kit of the invention comprises any antibody directed to immune checkpoint molecules; STING agonist; and/or chimeric protein used in methods of the present invention and/or pharmaceutical composition disclosed herein in unit dosage form. In embodiments, the unit dosage form is a container, such as a pre-filled syringe, which can be sterile, containing any agent disclosed herein and a pharmaceutically acceptable carrier, diluent, excipient, or vehicle. The kit can further comprise a label or printed instructions instructing the use of any agent disclosed herein. The kit may also include a lid speculum, topical anesthetic, and a cleaning agent for the administration location. In embodiments, the kit comprises a container containing an effective amount of a composition of the invention and an effective amount of another composition, such those disclosed herein.

Aspects of the present invention include use of a chimeric protein as disclosed herein in the manufacture of a medicament, e.g., a medicament for treatment of cancer and/or treatment of an inflammatory disease.

Methods of Selecting a Subject for Treatment and Evaluating the Efficacy of Cancer Treatment

In one aspect, the present disclosure relates to a method for evaluating the efficacy of cancer treatment in a subject in need thereof, wherein the subject is suffering from a cancer, the method comprising the steps of (i) providing the subject a pharmaceutical composition comprising a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain; (ii) obtaining a biological sample from the subject; (iii) performing an assay on the biological sample to determine level and/or activity of a mononuclear cells; and (iv) continuing administration of the heterologous chimeric protein if the subject has an increase in the level and/or activity of CD4⁺ cells, CD8⁺ cells, and/or NKP46⁺ NK cells.

In one aspect, the present disclosure relates to a method for evaluating the efficacy of cancer treatment in a subject in need thereof, wherein the subject is suffering from a cancer, the method comprising the steps of (i) providing the subject a pharmaceutical composition comprising (A) a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain; and (B) an anti-immune checkpoint antibody; (ii) obtaining a biological sample from the subject;

(iii) performing an assay on the biological sample to determine level and/or activity of a mononuclear cells; and (iv) continuing administration of the heterologous chimeric protein if the subject has an increase in the level and/or activity of CD4⁺ cells, CD8⁺ cells, and/or NKP46⁺ NK cells.

In one aspect, the present disclosure relates to a method of selecting a subject for treatment with a therapy for a cancer, the method comprising the steps of: (i) providing the subject a pharmaceutical composition comprising a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, (c) a linker linking the first domain and the second domain; (ii) obtaining a biological sample from the subject; (iii) performing an assay on the biological sample to determine level and/or activity of a mononuclear cells; and (iv) selecting the subject for treatment with the therapy for cancer if the subject has an increase in the level and/or activity of CD4⁺ cells, CD8⁺ cells, and/or NKP46⁺ NK cells.

In one aspect, the present disclosure relates to a method of selecting a subject for treatment with a therapy for a cancer, the method comprising the steps of: (i) providing the subject a pharmaceutical composition comprising (A) a heterologous chimeric protein comprising: (a) a first domain comprising a portion of the extracellular domain of T-cell immunoreceptor with Ig and ITIM domains (TIGIT), wherein the portion is capable of binding a TIGIT ligand, (b) a second domain comprising a portion of the extracellular domain of OX40L, wherein the portion is capable of binding a OX40L receptor, and (c) a linker linking the first domain and the second domain; and (B) optionally, an anti-immune checkpoint antibody; (ii) obtaining a biological sample from the subject; (iii) performing an assay on the biological sample to determine level and/or activity of a mononuclear cells; and (iv) selecting the subject for treatment with the therapy for cancer if the subject has an increase in the level and/or activity of CD4⁺ T cells, CD8⁺ cells, and/or NKP46⁺ NK cells.

In some embodiments, the increase in increase in the level and/or activity of CD4⁺ cells, CD8⁺ cells, and/or NKP46⁺ NK cells occurs by a factor of at least about 0.1×, about 0.2×, about 0.3×, about 0.4×, about 0.5×, about 0.6×, about 0.7×, about 0.8×, about 0.9×, about 1×, about 1.1×, about 1.2×, about 1.3×, about 1.4×, about 1.5×, about 1.6×, about 1.7×, about 1.8×, about 1.9×, about 2×, about 2.1×, about 2.2×, about 2.3×, about 2.4×, about 2.5×, about 2.6×, about 2.7×, about 2.8×, about 2.9×, about 3×, about 3.1×, about 3.2×, about 3.3×, about 3.4×, about 3.5×, about 3.6×, about 3.7×, about 3.8×, about 3.9×, about 4×, about 4.1×, about 4.2×, about 4.3×, about 4.4×, about 4.5×, about 4.6×, about 4.7×, about 4.8×, about 4.9×, about 5×, about 5.1×, about 5.2×, about 5.3×, about 5.4×, about 5.5×, about 5.6×, about 5.7×, about 5.8×, about 5.9×, about 6×, about 6.1×, about 6.2×, about 6.3×, about 6.4×, about 6.5×, about 6.6×, about 6.7×, about 6.8×, about 6.9×, about 7×, about 7.1×, about 7.2×, about 7.3×, about 7.4×, about 7.5×, about 7.6×, about 7.7×, about 7.8×, about 7.9×, about 8×, about 8.1×, about 8.2×, about 8.3×, about 8.4×, about 8.5×, about 8.6×, about 8.7×, about 8.8×, about 8.9×, about 9×, about 9.1×, about 9.2×, about 9.3×, about 9.4×, about 9.5×, about 9.6×, about 9.7×, about 9.8×, about 9.9×, or about 10× compared to the negative control.

In some embodiments, the increase is calculated in comparison to a level and/or activity of the cytokine in a positive control. In some embodiments, the positive control comprises the cytokine. In some embodiments, the positive control comprises the levels of the cytokine found in individuals that are undergoing an inflammatory response.

Additionally, or alternatively, in some embodiments, the subject has a decrease in the level and/or activity of at least one cytokine selected from IFNγ, IFNα, IL-27, CCL2, CCL3, CCL4, IL-2, TNFα, and IL-18. In some embodiments, the decrease is calculated in comparison to the level and/or activity of the cytokine in another biological sample in the subject prior to administering the dose of the chimeric protein to the subject. In some embodiments, the decrease is calculated in comparison to a level and/or activity of the cytokine in another biological sample from a different subject that has not been administered the dose of the chimeric protein. In some embodiments, the decrease is calculated in comparison to a level and/or activity of the cytokine in a negative control. In some embodiments, the negative control is devoid of the cytokine. In some embodiments, the negative control contains the levels of the cytokine found in individuals that are not undergoing an inflammatory response. In some embodiments, the decrease occurs by a factor of at least about 0.1×, about 0.2×, about 0.3×, about 0.4×, about 0.5×, about 0.6×, about 0.7×, about 0.8×, about 0.9×, about 1×, about 1.1×, about 1.2×, about 1.3×, about 1.4×, about 1.5×, about 1.6×, about 1.7×, about 1.8×, about 1.9×, about 2×, about 2.1×, about 2.2×, about 2.3×, about 2.4×, about 2.5×, about 2.6×, about 2.7×, about 2.8×, about 2.9×, about 3×, about 3.1×, about 3.2×, about 3.3×, about 3.4×, about 3.5×, about 3.6×, about 3.7×, about 3.8×, about 3.9×, about 4×, about 4.1×, about 4.2×, about 4.3×, about 4.4×, about 4.5×, about 4.6×, about 4.7×, about 4.8×, about 4.9×, about 5×, about 5.1×, about 5.2×, about 5.3×, about 5.4×, about 5.5×, about 5.6×, about 5.7×, about 5.8×, about 5.9×, about 6×, about 6.1×, about 6.2×, about 6.3×, about 6.4×, about 6.5×, about 6.6×, about 6.7×, about 6.8×, about 6.9×, about 7×, about 7.1×, about 7.2×, about 7.3×, about 7.4×, about 7.5×, about 7.6×, about 7.7×, about 7.8×, about 7.9×, about 8×, about 8.1×, about 8.2×, about 8.3×, about 8.4×, about 8.5×, about 8.6×, about 8.7×, about 8.8×, about 8.9×, about 9×, about 9.1×, about 9.2×, about 9.3×, about 9.4×, about 9.5×, about 9.6×, about 9.7×, about 9.8×, about 9.9×, or about 10× compared to the negative control.

In some embodiments, the decrease is calculated in comparison to a level and/or activity of the cytokine in a positive control. In some embodiments, the positive control comprises the cytokine. In some embodiments, the positive control comprises the levels of the cytokine found in individuals that are undergoing an inflammatory response.

In some embodiments of any of the aspects disclosed herein, the cancer is selected from the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, or urothelial carcinoma.

In some embodiments, the biological sample is a body fluid, a sample of separated cells, a sample from a tissue or an organ, or a sample of wash/rinse fluid obtained from an outer or inner body surface of a subject. In some embodiments, the biological sample is a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

In some embodiments, the biological sample is a fresh tissue sample, a frozen tumor tissue specimen, cultured cells, circulating tumor cells, or a formalin-fixed paraffin-embedded tumor tissue specimen. In some embodiments, the biological sample is the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, and urothelial carcinoma.

In some embodiments, the biological sample is obtained by a well known technique including, but not limited to scrapes, swabs or biopsies. In some embodiments, the biological sample is obtained by needle bipsy. In some embodiments, the biological sample is obtained by a technique selected from scrapes, swabs, and biopsy. In some embodiments, the biological sample is obtained by use of brushes, (cotton) swabs, spatula, rinse/wash fluids, punch biopsy devices, puncture of cavities with needles or surgical instrumentation. In some embodiments, the biological sample is or comprises cells obtained from an individual. In some embodiments, the obtained cells are or include cells from an individual from whom the biological sample is obtained. In some embodiments, a biological sample is a “primary sample” obtained directly from a source of interest by any appropriate means. For example, in some embodiments, the biological sample is obtained by methods selected from the group consisting of biopsy (e.g., fine needle aspiration or tissue biopsy), surgery, collection of body fluid (e.g., blood, lymph, feces etc.), etc. In some embodiments, the biological sample is originates from a tumor, blood, liver, the urogenital tract, the oral cavity, the upper aerodigestive tract the epidermis, or anal canal. It is to be understood that the biological sample may be further processed in order to carry out the method of the present technology. Such a “processed sample” may comprise, for example nucleic acids or proteins extracted from a sample or obtained by subjecting a primary sample to techniques such as amplification or reverse transcription of mRNA, isolation and/or purification of certain components, etc.

In some embodiments, the level and/or activity of CD4⁺ cells, CD8⁺ cells, and/or NKP46⁺ NK cells is measured by RNA sequencing, immunohistochemical staining, western blotting, in cell western, immunofluorescent staining, ELISA, and flow cytometry or a combination thereof. In some embodiments, the level and/or activity of CD4⁺ cells, CD8⁺ cells, and/or NKP46⁺ NK cells is measured by contacting the sample with an agent that specifically binds to one or more of the CD4⁺ cells, CD8⁺ cells, and/or NKP46⁺ NK cells. In some embodiments, the agent that specifically binds to one or more of the CD4±T cells, CD8⁺ cells, and/or NKP46⁺ NK cells is an antibody or fragment thereof. In some embodiments, the agent that specifically binds to one or more of the CD4⁺ cells, CD8⁺ cells, and/or NKP46⁺ NK cells is an antibody or fragment thereof. In some embodiments, the antibody is a recombinant antibody, a monoclonal antibody, a polyclonal antibody, or fragment thereof. In some embodiments, the antibody is specific to a surface marker selected from T-cell receptor, natural cytotoxicity receptor, CD3, CD4, CD8, CD16, CD30, CD40, CD38, CD57, CD127, NKP46, HLA-DR, perforin, granzyme, and granulysin.

In some embodiments, the level and/or activity of the cytokine is measured by one or more of RNA sequencing, immunohistochemical staining, western blotting, in cell western, immunofluorescent staining, ELISA, and flow cytometry.

In some embodiments, the level and/or activity of the cytokine is measured by contacting the sample with an agent that specifically binds to one or more of the cytokines. In some embodiments, the agent that specifically binds to one or more of the cytokines is an antibody or fragment thereof. In some embodiments, the antibody is a recombinant antibody, a monoclonal antibody, a polyclonal antibody, or fragment thereof. In some embodiments, the antibody is specific to a marker selected from T-cell receptor, natural cytotoxicity receptor, CD3, CD4, CD8, CD16, CD30, CD40, CD38, CD57, CD127, NKP46, HLA-DR, perforin, granzyme, and granulysin. In some embodiments, the antibody is specific to a tumor antigen.

In some embodiments, the level and/or activity of the cytokine is measured by contacting the sample with an agent that specifically binds to one or more of the nucleic acids. In some embodiments, the agent that specifically binds to one or more of the nucleic acids is a nucleic acid primer or probe. In some embodiments, the cytokine is selected from IFNγ, TNFα, IL-2, IL-4, IL-5, IL-6, IL-7, IL-9, IL-10, IL-13, IL-15, IL-17A, IL-17F, IL-22, CCL2, CCL3, CCL4, CXCL8, CXCL9, CXCL10, CXCL11 and CXCL12.

In some embodiments, the evaluating comprises prognosis, or response to treatment. In some embodiments, the evaluating comprises prognosis, and/or response to treatment. In some embodiments, the evaluating informs classifying the subject into a high or low risk group. In some embodiments, the high risk classification comprises a high level of cancer aggressiveness, wherein the aggressiveness is characterizable by one or more of a high tumor grade, low overall survival, high probability of metastasis, and the presence of a tumor marker indicative of aggressiveness. In some embodiments, the low risk classification comprises a low level of cancer aggressiveness, wherein the aggressiveness is characterizable by one or more of a low tumor grade, high overall survival, low probability of metastasis, and the absence and/or reduction of a tumor marker indicative of aggressiveness. In some embodiments, the low risk or high risk classification is indicative of withholding of neoadjuvant therapy. In some embodiments, the low risk or high risk classification is indicative of withholding of adjuvant therapy. In some embodiments, the low risk or high risk classification is indicative of continuing of the administration of the chimeric protein. In some embodiments, the low risk or high risk classification is indicative of withholding of the administration of the chimeric protein. In some embodiments, the low risk or high risk classification is indicative of continuing of the administration of the heterologous chimeric protein. In some embodiments, the low risk or high risk classification is indicative of withholding of the administration of the heterologous chimeric protein. In some embodiments, the evaluating is predictive of a positive response to and/or benefit from the administration of the heterologous chimeric protein. In some embodiments, the evaluating is predictive of a negative or neutral response to and/or benefit from the administration of the heterologous chimeric protein.

In some embodiments, the evaluating is predictive of a positive response to and/or benefit from the administration of the chimeric protein. In some embodiments, the evaluating is predictive of a negative or neutral response to and/or benefit from the administration of the chimeric protein. In some embodiments, the evaluating informs continuing the administration or withholding of the administration of the hererologous chimeric protein. In some embodiments, the evaluating informs continuing of the administration of the heterologous chimeric protein. In some embodiments, the evaluating informs changing the dose of the heterologous chimeric protein. In some embodiments, the evaluating informs increasing the dose of the heterologous chimeric protein. In some embodiments, the evaluating informs decreasing the dose of the heterologous chimeric protein. In some embodiments, the evaluating informs changing the regimen of administration of the heterologous chimeric protein. In some embodiments, the evaluating informs increasing the frequency of administration of the heterologous chimeric protein.

In some embodiments, the evaluating informs administration of neoadjuvant therapy. In some embodiments, the evaluating informs administration of adjuvant therapy. In some embodiments, the evaluating informs withholding of neoadjuvant therapy. In some embodiments, the evaluating informs changing of neoadjuvant therapy. In some embodiments, the evaluating informs changing of adjuvant therapy. In some embodiments, the evaluating informs withholding of adjuvant therapy.

In some embodiments, the evaluating is predictive of a positive response to and/or benefit from neoadjuvant chemotherapy or a non-responsiveness to and/or lack of benefit from neoadjuvant chemotherapy. In some embodiments, the evaluating is predictive of a positive response to and/or benefit from adjuvant chemotherapy or a non-responsiveness to and/or lack of benefit from adjuvant chemotherapy. In some embodiments, the evaluating is predictive of a negative or neutral response to and/or benefit from neoadjuvant chemotherapy or a non-responsiveness to and/or lack of benefit from neoadjuvant chemotherapy. In some embodiments, the evaluating is predictive of a negative or neutral response to and/or benefit from adjuvant chemotherapy or a non-responsiveness to and/or lack of benefit from adjuvant chemotherapy.

In some embodiments, the neoadjuvant therapy and/or adjuvant therapy is a chemotherapeutic agent. In some embodiments, the chemotherapeutic agent is selected from an alkylating agent, selected from thiotepa and CYTOXAN cyclosphosphamide; alkyl sulfonates, selected from busulfan, improsulfan and piposulfan; aziridines, selected from benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, selected from chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, selected from carmustine, chlorozotocin, fotemustine, lomustine, nimustine, and ranimnustine; antibiotics, selected from the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, selected from clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, selected from mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, selected from methotrexate and 5-fluorouracil (5-FU); folic acid analogues, selected from denopterin, methotrexate, pteropterin, trimetrexate; purine analogs, selected from fludarabine, 6-mercaptopurine, thiamiprine, thioguanine; pyrimidine analogs, selected from ancitabine, azacitidine, 6-azauridine, carmofur, cytarabine, dideoxyuridine, doxifluridine, enocitabine, floxuridine; androgens, selected from calusterone, dromostanolone propionate, epitiostanol, mepitiostane, testolactone; anti-adrenals, selected from minoglutethimide, mitotane, trilostane; folic acid replenisher, selected from frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids, selected from maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel, ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs, selected from cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids, selected from retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation, pharmaceutically acceptable salts, acids or derivatives thereof, and a combination of any two or more thereof.

In some embodiments, the neoadjuvant therapy and/or adjuvant therapy is a cytotoxic agent. In some embodiments, the cytotoxic agent is selected from methotrexate, aminopterin, 6-mercaptopurine, 6-thioguanine, cytarabine, 5-fluorouracil decarbazine; alkylating agents, selected from mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin, doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents, selected from the vinca alkaloids, vincristine and vinblastine, paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, pharmaceutically acceptable salts, acids or derivatives thereof, and a combination of any two or more thereof.

In some embodiments, the evaluating informs decreasing the frequency of administration of the chimeric protein.

In some embodiments, the neoadjuvant therapy and/or adjuvant therapy is checkpoint inhibitor. In some embodiments, the checkpoint inhibitor is an agent that targets one or more of TIM-3, BTLA, CTLA-4, B7-H4, GITR, galectin-9, HVEM, PD-L1, PD-L2, B7-H3, CD244, CD160, TIGIT, SIRPα, ICOS, CD172a, and TMIGD2.

Any aspect or embodiment disclosed herein can be combined with any other aspect or embodiment as disclosed herein.

The invention will be further described in the following examples, which do not limit the scope of the invention described in the claims.

### EXAMPLES

The examples herein are provided to illustrate advantages and benefits of the present technology and to further assist a person of ordinary skill in the art with practicing the method for treating a cancer of the present technology. The examples herein are also presented in order to more fully illustrate the certain aspects of the present technology. For example, the functional anti-tumor activity of specific combinations of antibodies directed to immune checkpoint molecules (e.g. anti-CTLA-4 and anti-PD-1) and a TIGIT-Fc-OX40L chimeric protein are illustrative embodiments. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1: Functional Anti-Tumor Activity of Anti-CTLA-4 Antibodies and a TIGIT-Fc-OX40L Chimeric Protein

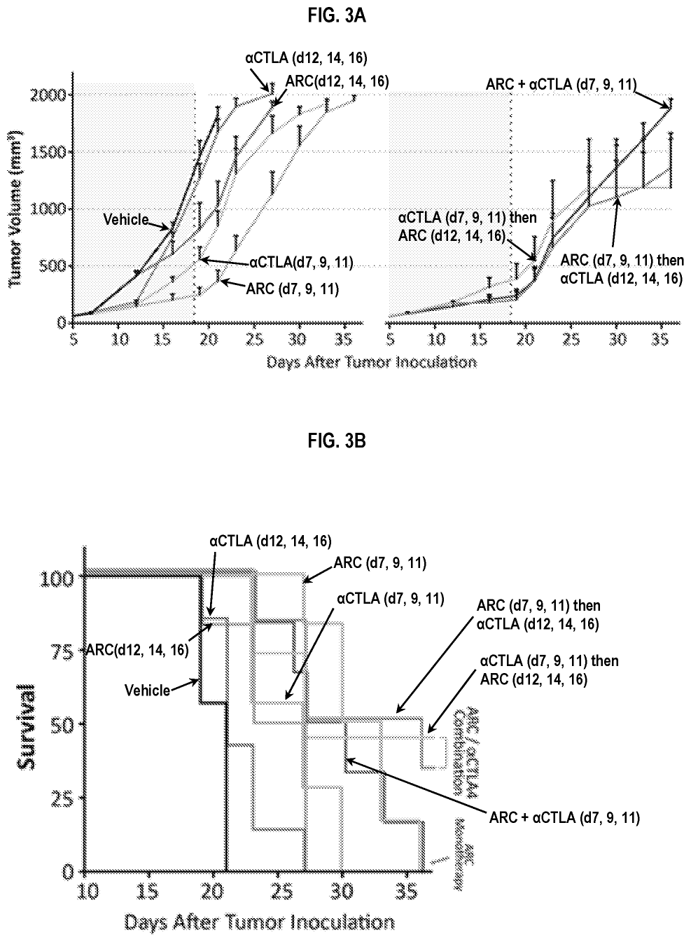

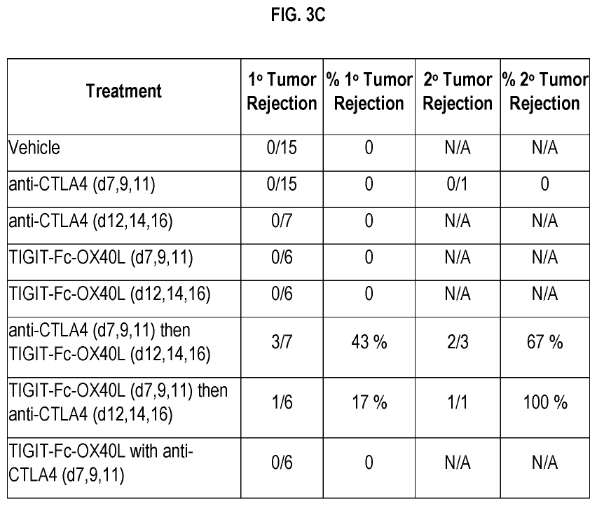

The in vivo ability of combinations of the TIGIT-Fc-OX40L chimeric protein with anti-CTLA-4 antibodies (FIG. 3A to FIG. 3C) to target and treat tumors were determined.

Mice were inoculated with tumors and were treated with a vehicle, an antibody, the TIGIT-Fc-OX40L chimeric protein, or combinations of the TIGIT-Fc-OX40L chimeric protein and an anti-CTLA-4 antibody; in the combinations, the TIGIT-Fc-OX40L chimeric protein was administered before the antibody, the TIGIT-Fc-OX40L chimeric protein was administered after the antibody, or the TIGIT-Fc-OX40L chimeric protein was administered with the antibody. See, FIG. 3A for the dose schedules. As shown in FIG. 3A, the following dose schedules were compared:
(1) Vehicle only;
(2) αCTLA antibody (administered on days 7, 9, and 11);
(3) αCTLA antibody (administered on days 12, 14, and 16);
(4) TIGIT-Fc-OX40L chimeric protein (ARC, administered on days 7, 9, and 11);
(5) TIGIT-Fc-OX40L chimeric protein (ARC, administered on days 12, 14, and 16);
(6) Combination of αCTLA antibody and TIGIT-Fc-OX40L chimeric protein (ARC), each administered on days 7, 9, and 11;
(7) Combination of αCTLA antibody and TIGIT-Fc-OX40L chimeric protein (ARC), where αCTLA antibody was administered on days 7, 9, and 11, and TIGIT-Fc-OX40L chimeric protein was administered on days 12, 14, and 16, and
(8) Combination of αCTLA antibody and TIGIT-Fc-OX40L chimeric protein (ARC), where TIGIT-Fc-OX40L chimeric protein was administered on days 7, 9, and 11, and αCTLA antibody was administered on days 12, 14, and 16.

FIG. 3A shows changes in tumor size (i.e., volume) resulting from treatments comprising the TIGIT-Fc-OX40L chimeric protein and/or the anti-CTLA-4 antibody. FIG. 3B shows Kaplan-Meier plots of the percent survival days after tumor inoculation resulting from treatments comprising the TIGIT-Fc-OX40L chimeric protein and/or the anti-CTLA-4 antibody. FIG. 3C includes data relevant to the graphs of FIG. 3A and FIG. 3B.

As shown in vehicle only-treated mice showed rapid development of tumors and lethality by day 21 (FIGS. 3A-3B). The mice that were administered αCTLA antibody on days 12, 14, and 16, showed slightly slower tumor development (FIG. 3A), and an extended survival (FIG. 3B) compared to the vehicle only-treated mice. The mice that were administered αCTLA antibody on days 7, 9, and 11, showed slower tumor development (FIG. 3A), and an extended survival compared to the vehicle only-treated mice, as well as the mice treated with αCTLA antibody on days 12, 14, and 16 (FIG. 3B).

The mice that were administered TIGIT-Fc-OX40L chimeric protein antibody on days 12, 14, and 16, showed slower tumor development (FIG. 3A), and an extended survival (FIG. 3B) compared to the vehicle only-treated mice. Likewise, the mice that were administered TIGIT-Fc-OX40L chimeric protein on days 7, 9, and 11, showed slower tumor development (FIG. 3A), and an extended survival (FIG. 3B) compared to the vehicle only-treated mice, and the mice treated with TIGIT-Fc-OX40L chimeric protein on days 12, 14, and 16. The mice that were administered TIGIT-Fc-OX40L chimeric protein showed slower tumor development (FIG. 3A) and an extended survival (FIG. 3B) compared to the dosing regimen-matched αCTLA antibody-treated mice.

The mice subjected to combination therapy of TIGIT-Fc-OX40L and αCTLA antibody, each administered on days 7, 9, and 11, showed slower tumor development (FIG. 3A), and an extended survival (FIG. 3B) compared to the mice subjected to monotherapy with either of TIGIT-Fc-OX40L and αCTLA antibody administered on days 7, 9, and 11. Further, the mice subjected to combination therapy of TIGIT-Fc-OX40L chimeric protein administered on days 7, 9, and 11 and αCTLA antibody administered on days 12, 14, and 16 showed slower tumor development (FIG. 3A), and an extended survival (FIG. 3B) compared to the mice subjected to monotherapy with either of TIGIT-Fc-OX40L and αCTLA antibody administered on days 7, 9, and 11. Also, the mice subjected to combination therapy with αCTLA antibody administered on days 7, 9, and 11 and TIGIT-Fc-OX40L chimeric protein administered on days 12, 14, and 16 showed slower tumor development (FIG. 3A), and an extended survival (FIG. 3B) compared to the mice subjected to monotherapy with either of TIGIT-Fc-OX40L and αCTLA antibody administered on days 7, 9, and 11. These results demonstrate that the combination of an αCTLA antibody and a TIGIT-Fc-OX40L heterologous chimeric protein show better antitumor activity compared to monotherapy with either of the αCTLA antibody and the TIGIT-Fc-OX40L heterologous chimeric protein. Accordingly, the combination therapy with an αCTLA antibody and a TIGIT-Fc-OX40L heterologous chimeric protein of the present technology is useful in the methods of treating cancer disclosed herein. These results also demonstrate that the αCTLA antibody and the TIGIT-Fc-OX40L heterologous chimeric protein may be administered simultaneously or one may be administered before the other.

Surprisingly, the mice subjected to combination therapy with a TIGIT-Fc-OX40L chimeric protein administered on days 7, 9, and 11 and an αCTLA antibody administered on days 12, 14, and 16 showed remarkably slower tumor development (FIG. 3A), and an extended survival (about 40% survival at day 37, see FIG. 3B) compared to the mice that were subjected to combination therapy of TIGIT-Fc-OX40L and αCTLA antibody simultaneously administered on days 7, 9, and 11. As shown in FIG. 3C, these mice exhibited 17% primary tumor rejection and 100% secondary tumor rejection. This is surprising because while the αCTLA antibody administered on days 12, 14, and 16 (in otherwise untreated mice) showed a very slight improvement in tumor growth and survival compared to vehicle only-treated mice (FIGS. 3A-3B) but the αCTLA antibody showed remarkable antitumor effect when administered on days 12, 14, and 16 to mice has undergone or is undergoing treatment with TIGIT-Fc-OX40L chimeric protein. Accordingly, an αCTLA antibody is useful in methods of treating cancer in a subject that has undergone or is undergoing treatment with TIGIT-Fc-OX40L heterologous chimeric protein of the present technology.

Surprisingly, the mice subjected to combination therapy with an αCTLA antibody administered on days 7, 9, and 11 and a TIGIT-Fc-OX40L chimeric protein administered on days 12, 14, and 16 showed remarkably slower tumor development (FIG. 3A), and an extended survival (about 30% survival at day 37, see FIG. 3B) compared to the mice that were subjected to combination therapy of TIGIT-Fc-OX40L and αCTLA antibody simultaneously administered on days 7, 9, and 11. As shown in FIG. 3C, these mice exhibited 43% primary tumor rejection and 67% secondary tumor rejection. Accordingly, the TIGIT-Fc-OX40L heterologous chimeric protein of the present technology is useful in methods of treating cancer in a subject that has undergone or is undergoing treatment with an αCTLA antibody.

### Example 2: Functional Anti-Tumor Activity of Anti-PD-1 Antibodies and a TIGIT-Fc-OX40L Chimeric Protein

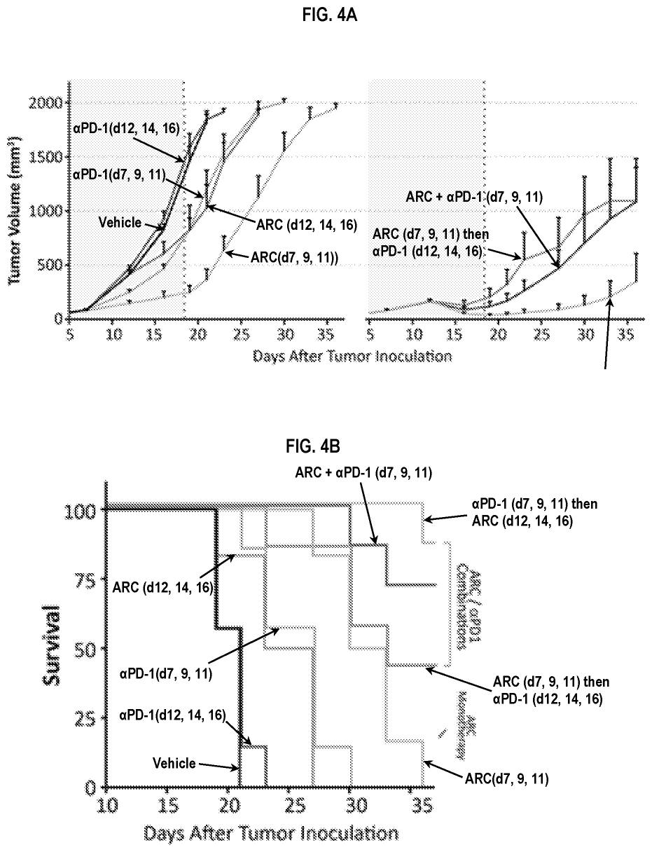

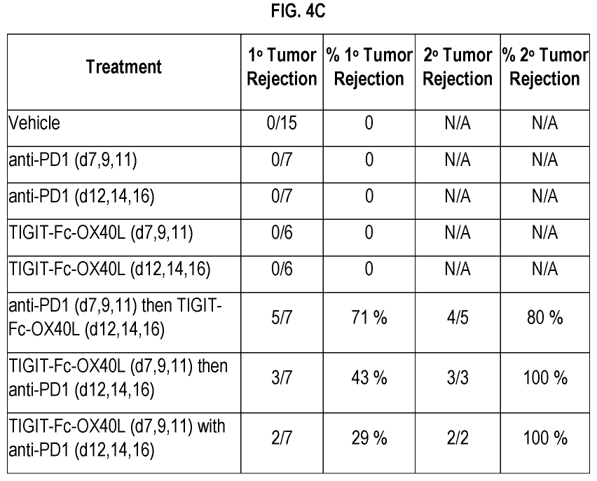

The in vivo ability of combinations of the TIGIT-Fc-OX40L chimeric protein with anti-PD-1 antibodies (FIG. 4A to FIG. 4D) to target and treat tumors were determined.

Mice were inoculated with tumors and were treated with a vehicle, an antibody, the TIGIT-Fc-OX40L chimeric protein, or combinations of the TIGIT-Fc-OX40L chimeric protein and an anti-PD-1 antibody; in the combinations, the TIGIT-Fc-OX40L chimeric protein was administered before the antibody, the TIGIT-Fc-OX40L chimeric protein was administered after the antibody, or the TIGIT-Fc-OX40L chimeric protein was administered with the antibody. As shown in FIG. 4A, the following dose schedules were compared:
(1) Vehicle only;
(2) αPD-1 antibody (administered on days 7, 9, and 11);
(3) αPD-1 antibody (administered on days 12, 14, and 16);
(4) TIGIT-Fc-OX40L chimeric protein (ARC, administered on days 7, 9, and 11);
(5) TIGIT-Fc-OX40L chimeric protein (ARC, administered on days 12, 14, and 16);
(6) Combination of αPD-1 antibody and TIGIT-Fc-OX40L chimeric protein (ARC), each administered on days 7, 9, and 11;
(7) Combination of αPD-1 antibody and TIGIT-Fc-OX40L chimeric protein (ARC), where αPD-1 antibody was administered on days 7, 9, and 11, and TIGIT-Fc-OX40L chimeric protein was administered on days 12, 14, and 16, and
(8) Combination of αPD-1 antibody and TIGIT-Fc-OX40L chimeric protein (ARC), where TIGIT-Fc-OX40L chimeric protein was administered on days 7, 9, and 11, and αPD-1 antibody was administered on days 12, 14, and 16.

FIG. 4A shows changes in tumor size (i.e., volume) resulting from treatments comprising the TIGIT-Fc-OX40L chimeric protein and/or the anti-CTLA-4 antibody. FIG. 4B shows Kaplan-Meier plots of the percent survival days after tumor inoculation resulting from treatments comprising the TIGIT-Fc-OX40L chimeric protein and/or the anti-CTLA-4 antibody. FIG. 4C and FIG. 4D include data relevant to the graphs of FIG. 4A and FIG. 4B.

As shown in vehicle only-treated mice showed rapid development of tumors and lethality by day 21 (FIGS. 4A-4B). The mice that were administered αPD-1 antibody on days 12, 14, and 16, showed a tumor development (FIG. 4A), and a survival (FIG. 4B) that was comparable with that of the vehicle only-treated mice. The mice that were administered αPD-1 antibody on days 7, 9, and 11, showed slower tumor development (FIG. 4A), and an extended survival compared to the vehicle only-treated mice, as well as the mice treated with αPD-1 antibody on days 12, 14, and 16 (FIG. 4B).

The mice that were administered TIGIT-Fc-OX40L chimeric protein antibody on days 12, 14, and 16, showed slower tumor development (FIG. 4A), and an extended survival (FIG. 4B) compared to the vehicle only-treated mice. Likewise, the mice that were administered TIGIT-Fc-OX40L chimeric protein on days 7, 9, and 11, showed slower tumor development (FIG. 4A), and an extended survival (FIG. 4B) compared to the vehicle only-treated mice, and the mice treated with TIGIT-Fc-OX40L chimeric protein on days 12, 14, and 16. The mice that were administered TIGIT-Fc-OX40L chimeric protein showed slower tumor development (FIG. 4A) and an extended survival (FIG. 4B) compared to the dosing regimen-matched αPD-1 antibody-treated mice.

Interestingly, the mice subjected to combination therapy of TIGIT-Fc-OX40L and αPD-1 antibody, each administered on days 7, 9, and 11, showed slower tumor development (FIG. 4A), and an extended survival (FIG. 4B) compared to the mice subjected to monotherapy with either of TIGIT-Fc-OX40L and αPD-1 antibody administered on days 7, 9, and 11. As shown in FIG. 4C, these mice exhibited 29% primary tumor rejection and 100% secondary tumor rejection. Further, the mice subjected to combination therapy of TIGIT-Fc-OX40L chimeric protein administered on days 7, 9, and 11 and αPD-1 antibody administered on days 12, 14, and 16 showed slower tumor development (FIG. 4A), and an extended survival (FIG. 4B) compared to the mice subjected to monotherapy with either of TIGIT-Fc-OX40L and αPD-1 antibody administered on days 7, 9, and 11. Also, the mice subjected to combination therapy with αPD-1 antibody administered on days 7, 9, and 11 and TIGIT-Fc-OX40L chimeric protein administered on days 12, 14, and 16 showed slower tumor development (FIG. 4A), and an extended survival (FIG. 4B) compared to the mice subjected to monotherapy with either of TIGIT-Fc-OX40L and αPD-1 antibody administered on days 7, 9, and 11. These results demonstrate that the combination of an αPD-1 antibody and a TIGIT-Fc-OX40L heterologous chimeric protein show better antitumor activity compared to monotherapy with either of the αPD-1 antibody and the TIGIT-Fc-OX40L heterologous chimeric protein. Accordingly, the combination therapy with an αPD-1 antibody and a TIGIT-Fc-OX40L heterologous chimeric protein of the present technology is useful in the methods of treating cancer disclosed herein. These results also demonstrate that the αPD-1 antibody and the TIGIT-Fc-OX40L heterologous chimeric protein may be administered simultaneously or one may be administered before the other.

Surprisingly, the mice subjected to combination therapy with a TIGIT-Fc-OX40L chimeric protein administered on days 7, 9, and 11 and an αPD-1 antibody administered on days 12, 14, and 16 showed remarkably slower tumor development (FIG. 4A), and an extended survival (about 40% survival at day 37, see FIG. 4B) compared to the mice that were subjected to monotherapy with either of TIGIT-Fc-OX40L and αPD-1 antibody. This is surprising because while the αPD-1 antibody administered on days 12, 14, and 16 (in otherwise untreated mice) showed a tumor growth and a survival comparable to vehicle only-treated mice (FIGS. 4A-4B) but the αPD-1 antibody showed remarkable antitumor effect when administered on days 12, 14, and 16 to mice has undergone or is undergoing treatment with TIGIT-Fc-OX40L chimeric protein. Moreover, as shown in FIG. 4C, these mice exhibited 43% primary tumor rejection and 100% secondary tumor rejection. Accordingly, an αPD-1 antibody is useful in methods of treating cancer in a subject that has undergone or is undergoing treatment with TIGIT-Fc-OX40L heterologous chimeric protein of the present technology.

Surprisingly, the mice subjected to combination therapy with an αPD-1 antibody administered on days 7, 9, and 11 and a TIGIT-Fc-OX40L chimeric protein administered on days 12, 14, and 16 showed remarkably slower tumor development (FIG. 4A), and an extended survival (about 30% survival at day 37, see FIG. 4B) compared to the mice that were subjected to combination therapy of TIGIT-Fc-OX40L and αPD-1 antibody simultaneously administered on days 7, 9, and 11. As shown in FIG. 4C, these mice exhibited 71% primary tumor rejection and 80% secondary tumor rejection. Accordingly, the TIGIT-Fc-OX40L heterologous chimeric protein of the present technology is useful in methods of treating cancer in a subject that has undergone or is undergoing treatment with an αPD-1 antibody.

FIG. 4A shows changes in tumor size (i.e., volume) resulting from treatments comprising the TIGIT-Fc-OX40L chimeric protein and/or the anti-PD-1 antibody. FIG. 4B shows Kaplan-Meier plots of the percent survival days after tumor inoculation resulting from treatments comprising the TIGIT-Fc-OX40L chimeric protein and/or the anti-PD-1 antibody. Surprisingly, the administration order of antibody and chimeric protein affected the treatment outcome. More specifically, although all of the combinations of chimeric protein and antibody provided an improved therapeutic benefit compared to any of the chimeric protein alone or the antibody alone treatments, among the combination treatments, the combination where the PD-1 antibody was administered before the TIGIT-Fc-OX40L chimeric protein had the greatest treatment outcome.

The experimental evidence shows that treatments with the TIGIT-Fc-OX40L chimeric protein and the anti-CTLA-4 antibody or treatments with the TIGIT-Fc-OX40L chimeric protein and the anti-PD-1 antibody provide most significant improvements in tumor volume and survival relative to treatments with the TIGIT-Fc-OX40L chimeric protein alone or either antibody alone.

### Example 3: Functional In Vivo Anti-Tumor Activity of Anti-PD1 Antibodies and a TIGIT-Fc-LIGHT Chimeric Protein

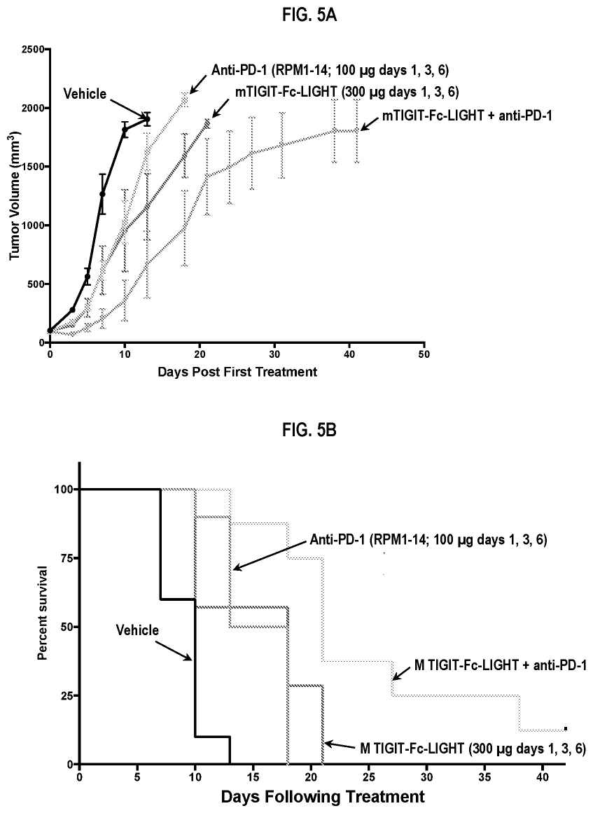

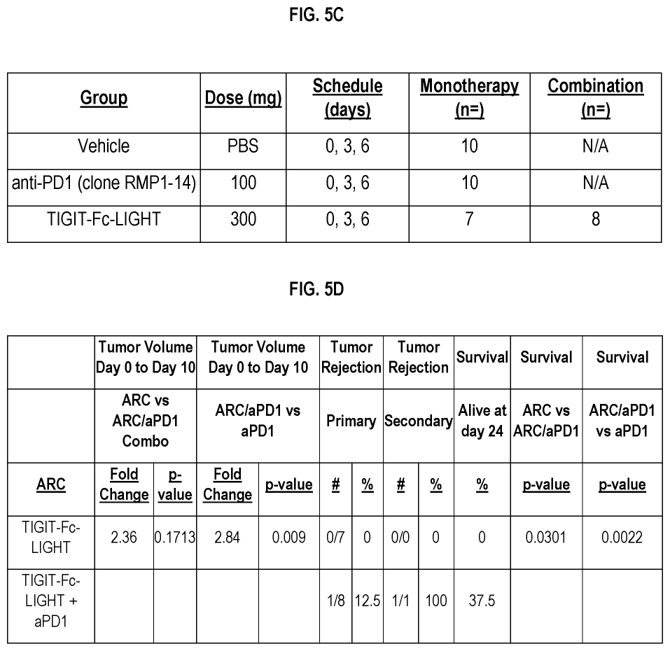

FIG. 5A shows tumor growth kinetics in a mouse challenged with CT26 tumor and treated as indicated in the legend (at day 10, the order of curves, top to bottom, is vehicle, anti-PD1, TIGIT-Fc-LIGHT, TIGIT-Fc-LIGHT+anti-PD1). The combination of the TIGIT-Fc-LIGHT chimeric protein and anti-PD1 antibody provided for the slowest tumor growth.

FIG. 5B is a Kaplan Meyer plot of survival and statistics of the CT26 tumor experiment of FIG. 5A. As shown, the combination of the TIGIT-Fc-LIGHT chimeric protein and anti-PD1 antibody was most effective as it provided 37.5% survival at day 24, as compared to 0% for monotherapies. FIG. 5C and FIG. 5D includes data relevant to the graphs of FIG. 5A and FIG. 5B.

As shown in FIG. 5A, the following dose schedules were compared:
(1) Vehicle only;
(2) anti-PD-1 (RPM1-14; 100 μg days 1, 3, and 6);
(3) mTIGIT-Fc-LIGHT chimeric protein (300 μg days 1, 3, 6); and
(4) Combination of anti-PD-1 antibody and mTIGIT-Fc-LIGHT chimeric protein (ARC), each administered on days 1, 3, and 6;

FIG. 5A shows tumor growth kinetics in a mouse challenged with CT26 tumor and treated as indicated in the legend (at day 10, the order of curves, top to bottom, is vehicle, anti-PD1, TIGIT-Fc-LIGHT, TIGIT-Fc-LIGHT+anti-PD1). The combination of the TIGIT-Fc-LIGHT chimeric protein and anti-PD1 antibody provided for the slowest tumor growth.

As shown in vehicle only-treated mice showed rapid development of tumors and lethality by day 13 (FIGS. 5A-5B). The mice that were administered αPD-1 antibody on days 1, 3, and 6 showed a slower tumor development (FIG. 5A), and an extended survival (FIG. 5B) compared to the vehicle only-treated mice. The mice that were administered TIGIT-Fc-LIGHT chimeric protein on days 1, 3, and 6, showed slower tumor development (FIG. 5A), and an extended survival compared to the vehicle only-treated mice, as well as the mice treated with αPD-1 antibody on days 12, 14, and 16 (FIG. 5B).

Interestingly, the mice subjected to combination therapy of TIGIT-Fc-LIGHT and the anti-PD-1 antibody, each administered on days 1, 3, and 6, showed slower tumor development (FIG. 5A), and an extended survival (FIG. 5B) compared to the mice subjected to monotherapy with either of TIGIT-Fc-LIGHT and the anti-PD-1 antibody administered on days 7, 9, and 11. As shown in FIG. 5D, the survival caused by the combination treatment was statistically significant over monotherapy with either of TIGIT-Fc-LIGHT (p=0.03) and the anti-PD-1 (p=0.0022). These results demonstrate that the combination of an αPD-1 antibody and a TIGIT-Fc-LIGHT heterologous chimeric protein show better antitumor activity compared to monotherapy with either of the αPD-1 antibody and the TIGIT-Fc-LIGHT heterologous chimeric protein. Accordingly, the combination therapy with an αPD-1 antibody and a TIGIT-Fc-LIGHT heterologous chimeric protein of the present technology is useful in the methods of treating cancer disclosed herein. These results also demonstrate that the αPD-1 antibody and the TIGIT-Fc-LIGHT heterologous chimeric protein may be administered simultaneously or one may be administered before the other.

### Example 4: Phenotyping of Tumor Infiltrating Lymphocytes in Animals Subjected to the Combination Therapy of the Present Technology

The above results demonstrate superior anti-tumor activity and increased survival when chimeric proteins (e.g. TIGIT-Fc-OX40L and TIGIT-Fc-LIGHT) are each combined with an anti-immune checkpoint antibody (e.g. anti-PD-1 and αCTLA antibody) as compared with each of the monotherapy. TIGIT is an inhibitory receptor on both T cells and natural killer (NK) cells. Without being bound by theory, TIGIT interaction with target receptors is thought to inhibit T/NK cytotoxic function. Without being bound by theory, TIGIT upregulation is thought to be one compensatory checkpoint mechanism observed in tumors refractory to anti-PD-1 therapies. Without being bound by theory, chimeric proteins such as TIGIT-Fc-OX40L and TIGIT-Fc-LIGHT can block this inhibitory interaction, allowing for anti-tumor immune responses following co-stimulation from the ARC co-stimulatory domain. Without being bound by theory, the above results suggest that combinations of chimeric proteins (e.g. TIGIT-Fc-OX40L and TIGIT-Fc-LIGHT) with anti-immune checkpoint antibodies (e.g. anti-PD-1 and αCTLA antibodies) have the potential to simultaneously block two checkpoint pathways (e.g. PD-1 and TIGIT, CTLA and TIGIT), while also providing immune co-stimulation (e.g. via OX40/L and LTbR/LIGHT).

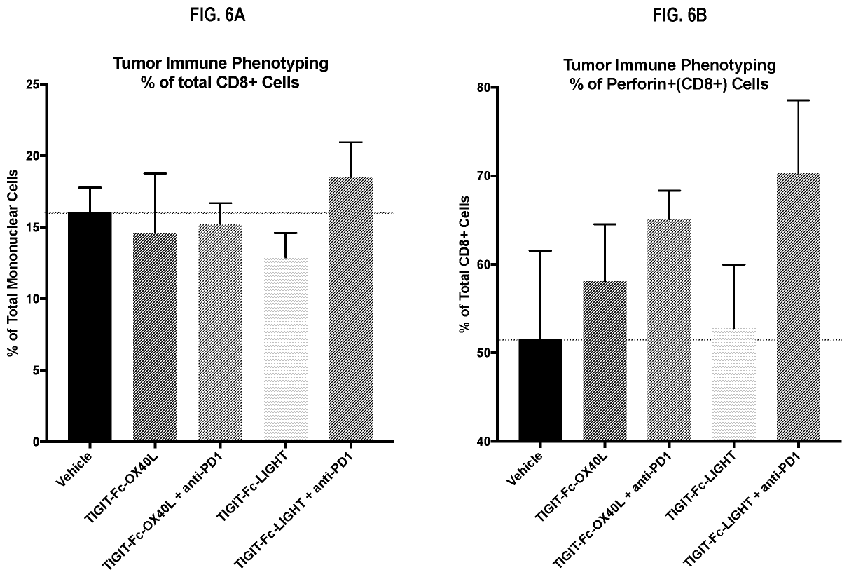

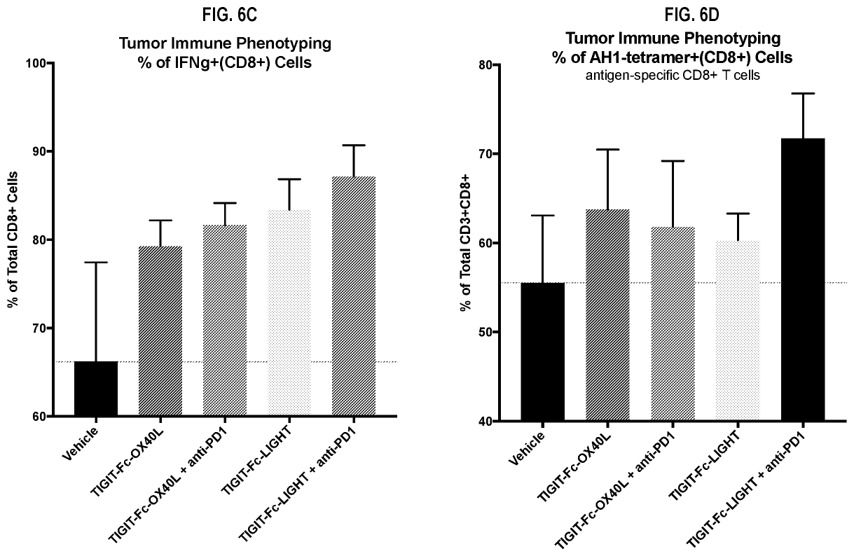

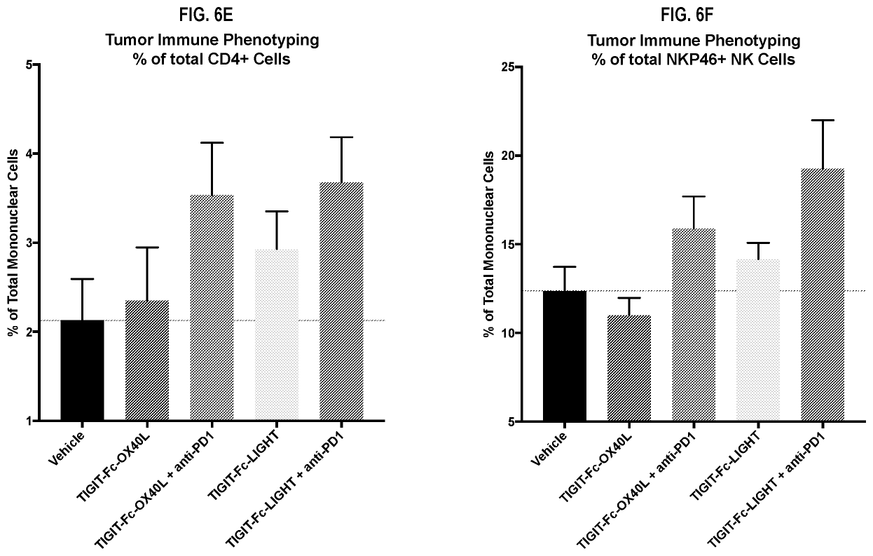

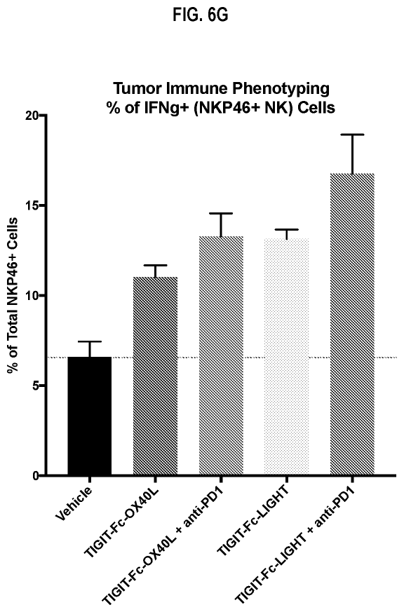

To further investigate the superior therapeutic effect of monotherapy with agonist redirected checkpoint (ARC) chimeric fusion proteins (e.g. TIGIT-Fc-OX40L and TIGIT-Fc-LIGHT) and combination therapy of the present technology (e.g. combinations of the chimeric fusion proteins (e.g. TIGIT-Fc-OX40L and TIGIT-Fc-LIGHT) with an anti-immune checkpoint antibody (e.g. anti-PD-1 and αCTLA antibody)), immune phenotyping of the tumor infiltrating lymphocytes (TIL) was performed. BALB/C mice were inoculated with CT26 (colorectal carcinoma) tumors, and when starting tumor volumes reached 30-60 mm³ (indicating day 0), treatment was begun. Mice were treated via intraperitoneal (IP) injection on days 0, 3, and 6 with 300 mg of either ARC (TIGIT-Fc-OX40L or TIGIT-Fc-LIGHT) or the combination of each ARC with 100 mg of anti-PD-1 (clone RMP1-14). On day 7, mice were euthanized, tumor tissue was collected and dissociated, and analyzed by flow cytometry and calculated as percent cell types. The total % of CD8+ T cells detected in the tumor are similar between treatment groups (FIG. 6A). As shown in FIG. 6B, the total Perforin CD8⁺ cells among the total CD8⁺ cells increased in mice treated with either TIGIT-Fc-OX40L or TIGIT-Fc-LIGHT chimeric fusion proteins. Combination treatment of the TIGIT-Fc-OX40L or TIGIT-Fc-LIGHT chimeric fusion proteins with anti-PD-1 antibody led to a further increase in Perforin CD8⁺ cells among the total CD8⁺ cell population (FIG. 6B). Similarly, as shown in FIG. 6C, the total IFNγ⁺ CD8⁺ cells increased among the total CD8⁺ cells in mice treated with either TIGIT-Fc-OX40L or TIGIT-Fc-LIGHT chimeric fusion proteins; and the combination treatment of the TIGIT-Fc-OX40L or TIGIT-Fc-LIGHT chimeric fusion proteins with anti-PD-1 antibody led to a further increase in IFNγ±CD8±cells among the total CD8⁺ cell population (FIG. 6C).

The immunodominant CD8+ T cell response against CT26 carcinoma cells is directed against a tumor/self-antigen, GP70423-431, also known as AH1. To evaluate the T cell response against CT26, total AH1-tetramer CD8⁺ cells were quantitated. As shown in FIG. 6D, the total AH1-tetramer CD8±cells increased among the total CD8⁺ cells in mice treated with either TIGIT-Fc-OX40L or TIGIT-Fc-LIGHT chimeric fusion proteins. Combination treatment of the TIGIT-Fc-OX40L or TIGIT-Fc-LIGHT chimeric fusion proteins with anti-PD-1 antibody led to a further increase in the total AH1-tetramer CD8±cells among the total CD8⁺ cell population (FIG. 6D). Collectively, these data show that while the total % of CD8+ T cells were not affected by any of the treatments, the proportion of the cells that are activated (as determined by expression of perforin, IFNγ, and tumor antigen specificity) were much higher when mice are treated with TIGIT ARCs; and increased further in most cases when ARCs are combined with anti-PD-1.

These results demonstrate that increased activation of CD8⁺ cells is correlated with the observed efficacy of combination treatment of chimeric proteins (e.g. TIGIT-Fc-OX40L and TIGIT-Fc-LIGHT) with anti-immune checkpoint antibodies (e.g. anti-PD-1 and αCTLA antibodies) of the present technology. Accordingly, measurements of activated T cells (e.g. by measuring total Perforin CD8⁺ cells, IFNγ±CD8±cells or tumor-specific T cells) is useful in the methods of selecting a subject for treatment with a therapy for a cancer disclosed herein.

Other relevant cell types, including total % of CD4+ T cells, NKP46⁺ NK cells, including the total IFNγ±NKP46⁺ NK cells in the tumors were also quantitated. As shown in FIG. 6E, the total CD4⁺ cells among the total mononuclear cells increased in mice treated with either TIGIT-Fc-OX40L or TIGIT-Fc-LIGHT chimeric fusion proteins. Combination treatment of the TIGIT-Fc-OX40L or TIGIT-Fc-LIGHT chimeric fusion proteins with anti-PD-1 antibody led to a further increase in CD4⁺ cells among the total mononuclear cell population (FIG. 6E). Similarly, as shown in FIG. 6F, the total NKP46⁺ NK cells increased among the total mononuclear cells in mice treated with either TIGIT-Fc-OX40L or TIGIT-Fc-LIGHT chimeric fusion proteins; and the combination treatment of the TIGIT-Fc-OX40L or TIGIT-Fc-LIGHT chimeric fusion proteins with anti-PD-1 antibody led to a further increase in NKP46⁺ NK cells among the mononuclear cell population (FIG. 6F). The IFNγ±cells were quantitated among NKP46⁺ NK cells. As shown in FIG. 6G, the total IFNγ±cells among the total NKP46⁺ NK cells increased in mice treated with either TIGIT-Fc-OX40L or TIGIT-Fc-LIGHT chimeric fusion proteins. Combination treatment of the TIGIT-Fc-OX40L or TIGIT-Fc-LIGHT chimeric fusion proteins with anti-PD-1 antibody led to a further increase in IFNγ±cells among the NKP46⁺ NK cell population (FIG. 6G).

These results demonstrate that increased numbers of CD4⁺ cells and NKP46⁺ NK cells, and the activated IFNγ±NKP46⁺ NK cells is correlated with the observed efficacy of combination treatment of chimeric proteins (e.g. TIGIT-Fc-OX40L and TIGIT-Fc-LIGHT) with anti-immune checkpoint antibodies (e.g. anti-PD-1 and αCTLA antibodies) of the present technology. Accordingly, measurements of CD4±T cells and NKP46⁺ NK cells, and the activated IFNγ±NKP46⁺ NK cells is useful in the methods of selecting a subject for treatment with a therapy for a cancer disclosed herein.

### Example 5: Functional In Vivo Anti-Tumor Activity of Specific Combinations of a STING Agonist and a TIGIT-Fc-OX40L Chimeric Protein

The in vivo ability of specific combinations of a stimulator of interferon genes (STING) agonist and the TIGIT-Fc-OX40L chimeric protein to target and reduce tumor volume are to be determined.

As in Example 1, above, mice are inoculated with tumors and treated with a vehicle, a STING Agonist, the TIGIT-Fc-OX40L chimeric protein, or combinations of the TIGIT-Fc-OX40L chimeric protein and a STING Agonist; in the combinations, the TIGIT-Fc-OX40L chimeric protein will be administered before the STING Agonist, the TIGIT-Fc-OX40L chimeric protein will be administered after the STING Agonist, or the TIGIT-Fc-OX40L chimeric protein will be administered with the STING Agonist. Dose schedules will be similar to those shown in FIG. 3A FIG. 3C, FIG. 4A, and FIG. 4C.

Illustrative STING agonists include, but are not limited to, 5,6-dimethylxanthenone-4-acetic acid (DMXAA), MIW815(ADU-S100), CRD5500, MK-1454, SB11285, IMSA101, and any STING agonist described in US20140341976, US20180028553, US20180230178, U.S. Pat. No. 9,549,944, WO2015185565, WO2016120305, WO2017044622, WO2017027645, WO2017027646, WO2017093933, WO2017106740, WO2017123657, WO2017123669, WO2017161349, WO2017175147, WO2017175156, WO2017176812, WO2018009466, WO2018045204, WO2018060323, WO2018098203, WO2018100558, WO2018138684, WO2018138685, WO2018152450, WO2018152453, WO2018172206, WO2018198084, WO2018234805, WO2018234807, WO2018234808, WO2019023459, WO2019046496, WO2019046498, WO2019046500, WO2019074887, WO2019079261, WO2019118839, WO2019125974, or WO2019160884, the contents of which are incorporated herein by reference in their entireties.

Tumor sizes will be assayed every other day until at least the 35th day after inoculation. Mice that reject the tumor will be re-challenged with a secondary tumor on the opposing flank, and primary/secondary tumors will continue to be measured.

The therapeutic activity of the treatments will be assayed. As examples, changes in tumor size (e.g., volume) and/or changes in survival of treated mice will be determined.

In any of the above-described Examples, the therapeutic activity of the treatments may further be assayed. In particular, changes in pharmacodynamic biomarkers showing tumor rejection will be determined by cytokine elevations in serum (in vivo) or changes in pharmacodynamic biomarkers in vitro in immune-related cells incubated with the super-antigen Staphylococcal enterotoxin B (SEB assay) or when cultured in AIM V media will be determined. Exemplary pharmacodynamic biomarkers include IFNγ, IL-2, IL-4, IL-5, IL-6, and IL-17A.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for treating a cancer in a subject in need thereof comprising:
providing the subject a first pharmaceutical composition comprising an antibody that is capable of binding cytotoxic T lymphocyte-associated antigen 4 (CTLA-4) and
providing the subject a second pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain.

2. The method of claim 1, wherein the first pharmaceutical composition and the second pharmaceutical composition are provided simultaneously.

3. The method of claim 1, wherein the first pharmaceutical composition is provided after the second pharmaceutical composition is provided.

4. The method of claim 1, wherein the first pharmaceutical composition is provided before the second pharmaceutical composition is provided.

5. The method of any one of claims 1 to 3, wherein the dose of the first pharmaceutical composition is less than the dose of the first pharmaceutical composition provided to a subject who has not undergone or is not undergoing treatment with the second pharmaceutical composition.

6. The method of any one of claim 1, 2, or 4, wherein the dose of the second pharmaceutical composition provided is less than the dose of the second pharmaceutical composition provided to a subject who has not undergone or is not undergoing treatment with the first pharmaceutical composition.

7. The method of any one of claims 1 to 6, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

8. The method of any one of claims 1 to 7, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

9. A method for treating a cancer in a subject comprising:
providing the subject a pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain;
wherein the subject has undergone or is undergoing treatment with an antibody that is capable of binding cytotoxic T lymphocyte-associated antigen 4 (CTLA-4).

10. The method of claim 9, wherein the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

11. The method of claim 9 or claim 10, wherein the subject has an increased chance of survival, a gain in weight, and/or a reduction in tumor size or cancer prevalence when compared to the subject who has not undergone or is not undergoing treatment with an antibody that is capable of binding PD-1 or binding a PD-1 ligand.

12. A method for treating a cancer in a subject comprising:
providing the subject a pharmaceutical composition comprising an antibody that is capable of binding cytotoxic T lymphocyte-associated antigen 4 (CTLA-4),
wherein the subject has undergone or is undergoing treatment with a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain.

13. The method of claim 12, wherein the dose of the pharmaceutical composition provided to the subject is less than the dose of the pharmaceutical composition that is provided to a subject who has not undergone or is not undergoing treatment with the heterologous chimeric protein.

14. The method of any one of claims 1 to 13, wherein the subject has a cancer that is poorly responsive or is refractory to treatment comprising the antibody that is capable of binding PD-1 or binding a PD-1 ligand.

15. The method of any one of claims 1 to 14, wherein the first domain comprises substantially all of the extracellular domain of TIGIT and/or the second domain comprises substantially all of the extracellular domain of OX40L.

16. The method of any one of claims 1 to 15, wherein the linker is a polypeptide selected from a flexible amino acid sequence, an IgG hinge region, and an antibody sequence.

17. The method of any one of claims 1 to 16, wherein the linker comprises at least one cysteine residue capable of forming a disulfide bond and/or comprises a hinge-CH2-CH3 Fc domain.

18. The method of claim 17, wherein the linker comprises a hinge-CH2-CH3 Fc domain derived from IgG1 or IgG4, e.g., human IgG1 or human IgG4.

19. The method of claim 17 or claim 18, wherein the linker comprises an amino acid sequence that is at least 95% identical to the amino acid sequence of SEQ ID NO: 1, SEQ ID NO: 2, or SEQ ID NO: 3.

20. The method of any one of claims 1 to 19, wherein the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, and urothelial carcinoma.

21. The method of any one of claims 1 to 20, wherein the subject has a cancer that is poorly responsive or is refractory to treatment comprising an antibody that is capable of binding PD-1 or binding a PD-1 ligand.

22. The method of any one of claims 1 to 21, wherein the cancer is poorly responsive or is non-responsive to treatment with an antibody that is capable of binding PD-1 or binding a PD-1 ligand after 12 weeks or so of such treatment.

23. The method of any one of claims 1 to 22, wherein the antibody that is capable of binding CTLA-4 is selected from the group consisting of YERVOY (ipilimumab), 9D9, tremelimumab (formerly ticilimumab, CP-675,206; MedImmune), AGEN1884, and RG2077.

24. A method for treating a cancer in a subject in need thereof comprising:
providing the subject a first pharmaceutical composition comprising an antibody that is capable of binding PD-1 or binding a PD-1 ligand and
providing the subject a second pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain.

25. The method of claim 24, wherein the first pharmaceutical composition and the second pharmaceutical composition are provided simultaneously.

26. The method of claim 24, wherein the first pharmaceutical composition is provided after the second pharmaceutical composition is provided.

27. The method of claim 24, wherein the first pharmaceutical composition is provided before the second pharmaceutical composition is provided.

28. The method of any one of claims 24 to 26, wherein the dose of the first pharmaceutical composition is less than the dose of the first pharmaceutical composition provided to a subject who has not undergone or is not undergoing treatment with the second pharmaceutical composition.

29. The method of any one of claim 24, 25, or 27, wherein the dose of the second pharmaceutical composition provided is less than the dose of the second pharmaceutical composition provided to a subject who has not undergone or is not undergoing treatment with the first pharmaceutical composition.

30. The method of any one of claims 24 to 29, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

31. The method of any one of claims 24 to 30, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

32. A method for treating a cancer in a subject comprising:
providing the subject a pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain;
wherein the subject has undergone or is undergoing treatment with an antibody that is capable of binding PD-1 or binding a PD-1 ligand.

33. The method of claim 32, wherein the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

34. The method of claim 32 or claim 10, wherein the subject has an increased chance of survival, a gain in weight, and/or a reduction in tumor size or cancer prevalence when compared to the subject who has not undergone or is not undergoing treatment with an antibody that is capable of binding PD-1 or binding a PD-1 ligand.

35. A method for treating a cancer in a subject comprising:
providing the subject a pharmaceutical composition comprising an antibody that is capable of binding PD-1 or binding a PD-1 ligand,
wherein the subject has undergone or is undergoing treatment with a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain.

36. The method of claim 35, wherein the dose of the pharmaceutical composition provided to the subject is less than the dose of the pharmaceutical composition that is provided to a subject who has not undergone or is not undergoing treatment with the heterologous chimeric protein.

37. The method of any one of claims 24 to 36, wherein the subject has a cancer that is poorly responsive or is refractory to treatment comprising the antibody that is capable of binding PD-1 or binding a PD-1 ligand.

38. The method of any one of claims 24 to 37, wherein the first domain comprises substantially all of the extracellular domain of TIGIT and/or the second domain comprises substantially all of the extracellular domain of OX40L.

39. The method of any one of claims 24 to 38, wherein the linker is a polypeptide selected from a flexible amino acid sequence, an IgG hinge region, and an antibody sequence.

40. The method of any one of claims 24 to 39, wherein the linker comprises at least one cysteine residue capable of forming a disulfide bond and/or comprises a hinge-CH2-CH3 Fc domain.

41. The method of claim 40, wherein the linker comprises a hinge-CH2-CH3 Fc domain derived from IgG1 or IgG4, e.g., human IgG1 or human IgG4.

42. The method of claim 40 or claim 41, wherein the linker comprises an amino acid sequence that is at least 95% identical to the amino acid sequence of SEQ ID NO: 1, SEQ ID NO: 2, or SEQ ID NO: 3.

43. The method of any one of claims 24 to 42, wherein the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, and urothelial carcinoma.

44. The method of any one of claims 24 to 43, wherein the subject has a cancer that is poorly responsive or is refractory to treatment comprising an antibody that is capable of binding PD-1 or binding a PD-1 ligand.

45. The method of any one of claims 24 to 44, wherein the cancer is poorly responsive or is non-responsive to treatment with an antibody that is capable of binding PD-1 or binding a PD-1 ligand after 12 weeks or so of such treatment.

46. The method of any one of claims 24 to 45, wherein the antibody that is capable of binding PD-1 or a PD-1 ligand is selected from the group consisting of nivolumab (ONO 4538, BMS 936558, MDX1106, OPDIVO (Bristol Myers Squibb)), pembrolizumab (KEYTRUDA/MK 3475, Merck), pidilizumab (CT 011, Cure Tech), RMP1-14, AGEN2034 (Agenus), and cemiplimab ((REGN-2810).

47. A method for treating a cancer in a subject in need thereof comprising:
providing the subject a first pharmaceutical composition comprising a stimulator of interferon genes (STING) agonist and
providing the subject a second pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain.

48. The method of claim 47, wherein the first pharmaceutical composition and the second pharmaceutical composition are provided simultaneously.

49. The method of claim 47, wherein the first pharmaceutical composition is provided after the second pharmaceutical composition is provided.

50. The method of claim 47, wherein the first pharmaceutical composition is provided before the second pharmaceutical composition is provided.

51. The method of any one of claims 47 to 49, wherein the dose of the first pharmaceutical composition is less than the dose of the first pharmaceutical composition provided to a subject who has not undergone or is not undergoing treatment with the second pharmaceutical composition.

52. The method of any one of claim 47, 48, or 50, wherein the dose of the second pharmaceutical composition provided is less than the dose of the second pharmaceutical composition provided to a subject who has not undergone or is not undergoing treatment with the first pharmaceutical composition.

53. The method of any one of claims 47 to 52, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

54. The method of any one of claims 47 to 53, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

55. A method for treating a cancer in a subject comprising:
providing the subject a pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain;
wherein the subject has undergone or is undergoing treatment with a stimulator of interferon genes (STING) agonist.

56. The method of claim 55, wherein the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

57. The method of claim 55 or claim 56, wherein the subject has an increased chance of survival, a gain in weight, and/or a reduction in tumor size or cancer prevalence when compared to the subject who has not undergone or is not undergoing treatment with an antibody that is capable of binding PD-1 or binding a PD-1 ligand.

58. A method for treating a cancer in a subject comprising:
providing the subject a pharmaceutical composition comprising a stimulator of interferon genes (STING) agonist,
wherein the subject has undergone or is undergoing treatment with a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor, and
(c) a linker linking the first domain and the second domain.

59. The method of claim 58, wherein the dose of the pharmaceutical composition provided to the subject is less than the dose of the pharmaceutical composition that is provided to a subject who has not undergone or is not undergoing treatment with the heterologous chimeric protein.

60. The method of any one of claims 47 to 59, wherein the subject has a cancer that is poorly responsive or is refractory to treatment comprising the antibody that is capable of binding PD-1 or binding a PD-1 ligand.

61. The method of any one of claims 47 to 60, wherein the first domain comprises substantially all of the extracellular domain of TIGIT and/or the second domain comprises substantially all of the extracellular domain of OX40L.

62. The method of any one of claims 47 to 61, wherein the linker is a polypeptide selected from a flexible amino acid sequence, an IgG hinge region, and an antibody sequence.

63. The method of any one of claims 47 to 62, wherein the linker comprises at least one cysteine residue capable of forming a disulfide bond and/or comprises a hinge-CH2-CH3 Fc domain.

64. The method of claim 63, wherein the linker comprises a hinge-CH2-CH3 Fc domain derived from IgG4, e.g., human IgG4.

65. The method of claim 63 or claim 64, wherein the linker comprises an amino acid sequence that is at least 95% identical to the amino acid sequence of SEQ ID NO: 1, SEQ ID NO: 2, or SEQ ID NO: 3.

66. The method of any one of claims 47 to 65, wherein the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, and urothelial carcinoma.

67. The method of any one of claims 47 to 66, wherein the subject has a cancer that is poorly responsive or is refractory to treatment comprising an antibody that is capable of binding PD-1 or binding a PD-1 ligand.

68. The method of any one of claims 47 to 67, wherein the cancer is poorly responsive or is non-responsive to treatment with an antibody that is capable of binding PD-1 or binding a PD-1 ligand after 12 weeks or so of such treatment.

69. The method of any one of claims 47 to 68, wherein the STING agonist is selected from the group consisting of 5,6-dimethylxanthenone-4-acetic acid (DMXAA), MIW815(ADU-S100), CRD5500, MK-1454, SB11285, and IMSA101.

70. A method for treating a cancer in a subject in need thereof comprising:
providing the subject a first pharmaceutical composition comprising an antibody that is capable of binding PD-1 or binding a PD-1 ligand and
providing the subject a second pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of LIGHT, wherein the portion is capable of binding a LIGHT receptor, and
(c) a linker linking the first domain and the second domain.

71. The method of claim 70, wherein the first pharmaceutical composition and the second pharmaceutical composition are provided simultaneously.

72. The method of claim 70, wherein the first pharmaceutical composition is provided after the second pharmaceutical composition is provided.

73. The method of claim 70, wherein the first pharmaceutical composition is provided before the second pharmaceutical composition is provided.

74. The method of any one of claims 70 to 73, wherein the dose of the first pharmaceutical composition is less than the dose of the first pharmaceutical composition provided to a subject who has not undergone or is not undergoing treatment with the second pharmaceutical composition.

75. The method of any one of claim 70, 71, or 73, wherein the dose of the second pharmaceutical composition provided is less than the dose of the second pharmaceutical composition provided to a subject who has not undergone or is not undergoing treatment with the first pharmaceutical composition.

76. The method of any one of claims 70 to 75, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

77. The method of any one of claims 70 to 76, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

78. A method for treating a cancer in a subject comprising:
providing the subject a pharmaceutical composition comprising a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of LIGHT, wherein the portion is capable of binding a LIGHT receptor, and
(c) a linker linking the first domain and the second domain;
wherein the subject has undergone or is undergoing treatment with an antibody that is capable of binding PD-1 or binding a PD-1 ligand.

79. The method of claim 78, wherein the dose of the pharmaceutical composition provided to the subject is less than the dose of the pharmaceutical composition that is provided to a subject who has not undergone or is not undergoing treatment with an antibody that is capable of binding PD-1 or binding a PD-1 ligand.

80. The method of claim 78, wherein the subject has an increased chance of survival, a gain in weight, and/or a reduction in tumor size or cancer prevalence when compared to the subject who has not undergone or is not undergoing treatment with an antibody that is capable of binding PD-1 or binding a PD-1 ligand.

81. A method for treating a cancer in a subject comprising:
providing the subject a pharmaceutical composition comprising an antibody that is capable of binding PD-1 or binding a PD-1 ligand,
wherein the subject has undergone or is undergoing treatment with a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of LIGHT, wherein the portion is capable of binding a LIGHT receptor, and
(c) a linker linking the first domain and the second domain.

82. The method of claim 81, wherein the dose of the pharmaceutical composition provided to the subject is less than the dose of the pharmaceutical composition that is provided to a subject who has not undergone or is not undergoing treatment with the heterologous chimeric protein.

83. The method of any one of claims 70 to 82, wherein the subject has a cancer that is poorly responsive or is refractory to treatment comprising the antibody that is capable of binding PD-1 or binding a PD-1 ligand.

84. The method of any one of claims 70 to 83, wherein the first domain comprises substantially all of the extracellular domain of TIGIT and/or the second domain comprises substantially all of the extracellular domain of LIGHT.

85. The method of any one of claims 70 to 84, wherein the linker is a polypeptide selected from a flexible amino acid sequence, an IgG hinge region, and an antibody sequence.

86. The method of any one of claims 70 to 85, wherein the linker comprises at least one cysteine residue capable of forming a disulfide bond and/or comprises a hinge-CH2-CH3 Fc domain.

87. The method of claim 86, wherein the linker comprises a hinge-CH2-CH3 Fc domain derived from IgG1 or IgG4, e.g., human IgG1 or human IgG4.

88. The method of claim 86 or claim 87, wherein the linker comprises an amino acid sequence that is at least 95% identical to the amino acid sequence of SEQ ID NO: 1, SEQ ID NO: 2, or SEQ ID NO: 3.

89. The method of any one of claims 70 to 88, wherein the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, and urothelial carcinoma.

90. The method of any one of claims 70 to 89, wherein the subject has a cancer that is poorly responsive or is refractory to treatment comprising an antibody that is capable of binding PD-1 or binding a PD-1 ligand.

91. The method of any one of claims 70 to 90, wherein the cancer is poorly responsive or is non-responsive to treatment with an antibody that is capable of binding PD-1 or binding a PD-1 ligand after 12 weeks or so of such treatment.

92. The method of any one of claims 70 to 91, wherein the antibody that is capable of binding PD-1 or a PD-1 ligand is selected from the group consisting of nivolumab (ONO 4538, BMS 936558, MDX1106, OPDIVO (Bristol Myers Squibb)), pembrolizumab (KEYTRUDA/MK 3475, Merck), pidilizumab (CT 011, Cure Tech), RMP1-14, AGEN2034 (Agenus), and cemiplimab ((REGN-2810).

93. A method for evaluating the efficacy of cancer treatment in a subject in need thereof, wherein the subject is suffering from a cancer, the method comprising the steps of
(i) providing the subject a pharmaceutical composition comprising a heterologous chimeric protein comprising:
(A) a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor or a portion of the extracellular domain of LIGHT, wherein the portion is capable of binding a LIGHT receptor,
(c) a linker linking the first domain and the second domain; and
(B) optionally, an anti-immune checkpoint antibody;
(ii) obtaining a biological sample from the subject;
(iii) performing an assay on the biological sample to determine level and/or activity of a mononuclear cells; and
(iv) continuing administration of the heterologous chimeric protein if the subject has an increase in the level and/or activity of CD4⁺ T cells, CD8⁺ T cells, and/or NKP46+NK cells.

94. A method of selecting a subject for treatment with a therapy for a cancer, the method comprising the steps of:
(i) providing the subject a pharmaceutical composition comprising
(A) a heterologous chimeric protein comprising:
(a) a first domain comprising a portion of the extracellular domain of T-cell immunoreceptor with Ig and ITIM domains (TIGIT), wherein the portion is capable of binding a TIGIT ligand,
(b) a second domain comprising a portion of the extracellular domain of OX40L, wherein the portion is capable of binding a OX40L receptor or a portion of the extracellular domain of LIGHT, wherein the portion is capable of binding a LIGHT receptor,
(c) a linker linking the first domain and the second domain; and
(B) optionally, an anti-immune checkpoint antibody;
(ii) obtaining a biological sample from the subject;
(iii) performing an assay on the biological sample to determine level and/or activity of a mononuclear cells; and
(iv) selecting the subject for treatment with the therapy for cancer if the subject has an increase in the level and/or activity of CD4⁺ T cells, CD8⁺ T cells, and/or NKP46+NK cells.

95. The method of claim 93 or claim 94, wherein the cancer is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, or urothelial carcinoma.

96. The method of any one of claims 93-95, wherein the biological sample is a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

97. The method of any one of claims 93-96, wherein the biological sample is a fresh tissue sample, a frozen tumor tissue specimen, cultured cells, circulating tumor cells, or a formalin-fixed paraffin-embedded tumor tissue specimen.

98. The method of any one of claims 93-97, wherein the biological sample is a tumor sample derived from a tumor that is or is related to a cancer selected from Hodgkin's lymphoma, non-Hodgkin's lymphoma, adrenal cancer, anal cancer, breast cancer, cervical cancer, cholangiocarcinoma, colorectal cancer, endometrial cancer, esophageal cancer, gastric cancer, head and neck cancer, hepatocellular carcinoma, melanoma, Merkel cell carcinoma, non-small cell lung cancer, prostate cancer, pancreatic cancer, renal cell carcinoma, ovarian (including fallopian tube and peritoneal cancers) cancer, small cell lung cancer, squamous cell carcinoma of the skin, sarcomas, thyroid cancers, or urothelial carcinoma.

99. The method of any one of claims 93-98, wherein the biological sample is obtained by a technique selected from scrapes, swabs, and biopsy.

100. The method of any one of claims 93-99, wherein the biological sample is obtained by use of brushes, (cotton) swabs, spatula, rinse/wash fluids, punch biopsy devices, puncture of cavities with needles or surgical instrumentation.

101. The method of any one of claims 93-100, wherein the level and/or activity of CD4⁺ T cells, CD8⁺ T cells, and/or NKP46⁺ NK cells is measured by RNA sequencing, immunohistochemical staining, western blotting, in cell western, immunofluorescent staining, ELISA, and flow cytometry or a combination thereof.

102. The method of any one of claims 93-101, wherein the level and/or activity of CD4⁺ T cells, CD8⁺ T cells, and/or NKP46⁺ NK cells is measured by contacting the sample with an agent that specifically binds to one or more of the CD4⁺ T cells, CD8⁺ T cells, and/or NKP46⁺ NK cells.

103. The method of claim 101, wherein the agent that specifically binds to one or more of the CD4⁺ T cells, CD8⁺ T cells, and/or NKP46⁺ NK cells is an antibody or fragment thereof.

104. The method of claim 101, wherein the agent that specifically binds to one or more of the CD4⁺ T cells, CD8⁺ T cells, and/or NKP46⁺ NK cells is an antibody or fragment thereof.

105. The method of claim 104, wherein the antibody is a recombinant antibody, a monoclonal antibody, a polyclonal antibody, or fragment thereof.

106. The method of claim 104 or claim 105, wherein the antibody is specific to a marker selected from T-cell receptor, natural cytotoxicity receptor, CD3, CD4, CD8, CD16, CD30, CD40, CD38, CD57, CD127, NKP46, HLA-DR, perforin, granzyme, and granulysin.

107. The method of claim 104 or claim 105, wherein the antibody is specific to a tumor antigen.

108. The method of any one of claims 93-107, wherein the level and/or activity of the cytokine is measured by contacting the sample with an agent that specifically binds to one or more of the nucleic acids.

109. The method of claim 108, wherein the agent that specifically binds to one or more of the nucleic acids is a nucleic acid primer or probe.

110. The method of claim 108 or claim 109, wherein the agent that specifically binds to one or more of the nucleic acids that is homologous or complimentary an mRNA encoding a cytokine.

111. The method of claim 110, wherein the cytokine is selected from IFNγ, TNFα, IL-2, IL-4, IL-5, IL-6, IL-7, IL-9, IL-10, IL-13, IL-15, IL-17A, IL-17F, IL-22, CCL2, CCL3, CCL4, CXCL8, CXCL9, CXCL10, CXCL11 and CXCL12.

112. The method of any one of claims 93 or 95 to 111, wherein the evaluating comprises prognosis, or response to treatment.

113. The method of any one of claims 93 or 95 to 112, wherein the evaluating informs classifying the subject into a high or low risk group.

114. The method of claim 113, wherein the high risk classification comprises a high level of cancer aggressiveness, wherein the aggressiveness is characterizable by one or more of a high tumor grade, low overall survival, high probability of metastasis, and the presence of a tumor marker indicative of aggressiveness.

115. The method of claim 113 or claim 114, wherein the low risk classification comprises a low level of cancer aggressiveness, wherein the aggressiveness is characterizable by one or more of a low tumor grade, high overall survival, low probability of metastasis, and the absence and/or reduction of a tumor marker indicative of aggressiveness.

116. The method of any one of claims 113 to 115, wherein the low risk or high risk classification is indicative of withholding of neoadjuvant therapy.

117. The method of any one of claims 113 to 116, wherein the low risk or high risk classification is indicative of withholding of adjuvant therapy.

118. The method of any one of claims 113 to 117, wherein the low risk or high risk classification is indicative of continuing of the administration of the heterologous chimeric protein.

119. The method of any one of claims 113 to 118, wherein the low risk or high risk classification is indicative of withholding of the administration of the heterologous chimeric protein.

120. The method of any one of claims 93 or 95 to 119, wherein the evaluating is predictive of a positive response to and/or benefit from the administration of the heterologous chimeric protein.

121. The method of any one of claims 93 or 95 to 120, wherein the evaluating is predictive of a negative or neutral response to and/or benefit from the administration of the heterologous chimeric protein.

122. The method of any one of claims 93 or 95 to 121, wherein the evaluating informs continuing the administration or withholding of the administration of the heterologous chimeric protein.

123. The method of claim 122, wherein the evaluating informs continuing of the administration of the heterologous chimeric protein.

124. The method of any one of claims 93 or 95 to 123, wherein the evaluating informs administration of neoadjuvant therapy.

125. The method of any one of claims 93 or 95 to 124, wherein the evaluating informs withholding of neoadjuvant therapy.

126. The method of any one of claims 93 or 95 to 125, wherein the evaluating informs administration of adjuvant therapy.

127. The method of any one of claims 93 or 95 to 126, wherein the evaluating informs changing of neoadjuvant therapy.

128. The method of any one of claims 93 or 95 to 127, wherein the evaluating informs changing of adjuvant therapy.

129. The method of any one of claims 93 or 95 to 128, wherein the evaluating informs withholding of adjuvant therapy.

130. The method of any one of claims 93 or 95 to 129, wherein the evaluating is predictive of a positive response to and/or benefit from neoadjuvant chemotherapy or a non-responsiveness to and/or lack of benefit from neoadjuvant chemotherapy.

131. The method of any one of claims 93 or 95 to 130, wherein the evaluating is predictive of a negative or neutral response to and/or benefit from neoadjuvant chemotherapy or a non-responsiveness to and/or lack of benefit from neoadjuvant chemotherapy.

132. The method of any one of claims 93 or 95 to 131, wherein the evaluating is predictive of a positive response to and/or benefit from adjuvant chemotherapy or a non-responsiveness to and/or lack of benefit from adjuvant chemotherapy.

133. The method of any one of claims 93 or 95 to 132, wherein the evaluating is predictive of a negative or neutral response to and/or benefit from adjuvant chemotherapy or a non-responsiveness to and/or lack of benefit from adjuvant chemotherapy.

134. The method of any one of claims 116 to 133, wherein the neoadjuvant therapy and/or adjuvant therapy is a chemotherapeutic agent.

135. The method of claim 134, wherein the chemotherapeutic agent is selected from an alkylating agent, selected from thiotepa and CYTOXAN cyclosphosphamide; alkyl sulfonates, selected from busulfan, improsulfan and piposulfan; aziridines, selected from benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, selected from chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, selected from carmustine, chlorozotocin, fotemustine, lomustine, nimustine, and ranimnustine; antibiotics, selected from the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, selected from clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, selected from mitomycin C, mycophenolic acid, nogalamycin, olivomycins, peplomycin, poffiromycin, puromycin, quelamycin, rodorubicin, streptonigrin, streptozocin, tubercidin, ubenimex, zinostatin, zorubicin; anti-metabolites, selected from methotrexate and 5-fluorouracil (5-FU); folic acid analogues, selected from denopterin, methotrexate, pteropterin, trimetrexate; purine analogs, selected from fludarabine, 6-mercaptopurine, thiamiprine, thioguanine; pyrimidine analogs, selected from ancitabine, azacitidine, 6-azauridine, carmofur, cytarabine, dideoxyuridine, doxifluridine, enocitabine, floxuridine; androgens, selected from calusterone, dromostanolone propionate, epitiostanol, mepitiostane, testolactone; anti-adrenals, selected from minoglutethimide, mitotane, trilostane; folic acid replenisher, selected from frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids, selected from maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel, ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs, selected from cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids, selected from retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation, pharmaceutically acceptable salts, acids or derivatives thereof, and a combination of any two or more thereof.

136. The method of any one of claims 116 to 135, wherein the neoadjuvant therapy and/or adjuvant therapy is a cytotoxic agent.

137. The method of claim 136, wherein the cytotoxic agent is selected from methotrexate, aminopterin, 6-mercaptopurine, 6-thioguanine, cytarabine, 5-fluorouracil decarbazine; alkylating agents, selected from mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin, doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents, selected from the vinca alkaloids, vincristine and vinblastine, paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, pharmaceutically acceptable salts, acids or derivatives thereof, and a combination of any two or more thereof.

138. The method of any one of claims 116 to 137, wherein the neoadjuvant therapy and/or adjuvant therapy is checkpoint inhibitor. In some embodiments, the checkpoint inhibitor is an agent that targets one of TIM-3, BTLA, CTLA-4, B7-H4, GITR, galectin-9, HVEM, PD-L1, PD-L2, B7-H3, CD244, CD160, TIGIT, SIRPα, ICOS, CD172a, and TMIGD2.


SEQUENCE LISTING (61 sequences)

SEQ ID NO: 1 - protein, 217 aa, Artificial Sequence
  Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic Polypeptide
SKYGPPCPSCP

SEQ ID NO: 5 - protein, 11 aa, Artificial Sequence
  Synthetic Polypeptide
SKYGPPCPPCP

SEQ ID NO: 6 - protein, 6 aa, Artificial Sequence
  Synthetic Polypeptide
SKYGPP

SEQ ID NO: 7 - protein, 6 aa, Artificial Sequence
  Synthetic Polypeptide
IEGRMD

SEQ ID NO: 8 - protein, 9 aa, Artificial Sequence
  Synthetic Polypeptide
GGGVPRDCG

SEQ ID NO: 9 - protein, 15 aa, Artificial Sequence
  Synthetic Polypeptide
IEGRMDGGGGAGGGG

SEQ ID NO: 10 - protein, 8 aa, Artificial Sequence
  Synthetic Polypeptide
GGGSGGGS

SEQ ID NO: 11 - protein, 12 aa, Artificial Sequence
  Synthetic Polypeptide
GGGSGGGGSGGG

SEQ ID NO: 12 - protein, 14 aa, Artificial Sequence
  Synthetic Polypeptide
EGKSSGSGSESKST

SEQ ID NO: 13 - protein, 4 aa, Artificial Sequence
  Synthetic Polypeptide
GGSG

SEQ ID NO: 14 - protein, 12 aa, Artificial Sequence
  Synthetic Polypeptide
GGSGGGSGGGSG

SEQ ID NO: 15 - protein, 15 aa, Artificial Sequence
  Synthetic Polypeptide
EAAAKEAAAKEAAAK

SEQ ID NO: 16 - protein, 20 aa, Artificial Sequence
  Synthetic Polypeptide
EAAAREAAAREAAAREAAAR

SEQ ID NO: 17 - protein, 17 aa, Artificial Sequence
  Synthetic Polypeptide
GGGGSGGGGSGGGGSAS

SEQ ID NO: 18 - protein, 9 aa, Artificial Sequence
  Synthetic Polypeptide
GGGGAGGGG

SEQ ID NO: 19 - skipped

SEQ ID NO: 20 - protein, 6 aa, Artificial Sequence
  Synthetic Polypeptide
GSGSGS

SEQ ID NO: 21 - protein, 10 aa, Artificial Sequence
  Synthetic Polypeptide
GSGSGSGSGS

SEQ ID NO: 22 - protein, 7 aa, Artificial Sequence
  Synthetic Polypeptide
GGGGSAS

SEQ ID NO: 23 - protein, 20 aa, Artificial Sequence
  Synthetic Polypeptide
APAPAPAPAPAPAPAPAPAP

SEQ ID NO: 24 - protein, 4 aa, Artificial Sequence
  Synthetic Polypeptide
CPPC

SEQ ID NO: 25 - protein, 5 aa, Artificial Sequence
  Synthetic Polypeptide
GGGGS

SEQ ID NO: 26 - protein, 10 aa, Artificial Sequence
  Synthetic Polypeptide
GGGGSGGGGS

SEQ ID NO: 27 - protein, 15 aa, Artificial Sequence
  Synthetic Polypeptide
GGGGSGGGGSGGGGS

SEQ ID NO: 28 - protein, 20 aa, Artificial Sequence
  Synthetic Polypeptide
GGGGSGGGGSGGGGSGGGGS

SEQ ID NO: 29 - protein, 25 aa, Artificial Sequence
  Synthetic Polypeptide
GGGGSGGGGSGGGGSGGGGSGGGGS

SEQ ID NO: 30 - protein, 30 aa, Artificial Sequence
  Synthetic Polypeptide
GGGGSGGGGSGGGGSGGGGSGGGGSGGGGS

SEQ ID NO: 31 - protein, 35 aa, Artificial Sequence
  Synthetic Polypeptide
GGGGSGGGGSGGGGSGGGGSGGGGSGGGGSGGGGS

SEQ ID NO: 32 - protein, 40 aa, Artificial Sequence
  Synthetic Polypeptide
GGGGSGGGGSGGGGSGGGGSGGGGSGGGGSGGGGSGGGGS

SEQ ID NO: 33 - protein, 16 aa, Artificial Sequence
  Synthetic Polypeptide
GGSGGSGGGGSGGGGS

SEQ ID NO: 34 - protein, 8 aa, Artificial Sequence
  Synthetic Polypeptide
GGGGGGGG

SEQ ID NO: 35 - protein, 6 aa, Artificial Sequence
  Synthetic Polypeptide
GGGGGG

SEQ ID NO: 36 - protein, 5 aa, Artificial Sequence
  Synthetic Polypeptide
EAAAK

SEQ ID NO: 37 - protein, 10 aa, Artificial Sequence
  Synthetic Polypeptide
EAAAKEAAAK

SEQ ID NO: 38 - protein, 15 aa, Artificial Sequence
  Synthetic Polypeptide
EAAAKEAAAKEAAAK

SEQ ID NO: 39 - protein, 12 aa, Artificial Sequence
  Synthetic Polypeptide
AEAAAKEAAAKA

SEQ ID NO: 40 - protein, 17 aa, Artificial Sequence
  Synthetic Polypeptide
AEAAAKEAAAKEAAAKA

SEQ ID NO: 41 - protein, 22 aa, Artificial Sequence
  Synthetic Polypeptide
AEAAAKEAAAKEAAAKEAAAKA

SEQ ID NO: 42 - protein, 27 aa, Artificial Sequence
  Synthetic Polypeptide
AEAAAKEAAAKEAAAKEAAAKEAAAKA

SEQ ID NO: 43 - protein, 46 aa, Artificial Sequence
  Synthetic Polypeptide
AEAAAKEAAAKEAAAKEAAAKALEAEAAAKEAAAKEAAAKEAAAKA

SEQ ID NO: 44 - protein, 5 aa, Artificial Sequence
  Synthetic Polypeptide
PAPAP

SEQ ID NO: 45 - protein, 18 aa, Artificial Sequence
  Synthetic Polypeptide
KESGSVSSEQLAQFRSLD

SEQ ID NO: 46 - protein, 12 aa, Artificial Sequence
  Synthetic Polypeptide
GSAGSAAGSGEF

SEQ ID NO: 47 - protein, 5 aa, Artificial Sequence
  Synthetic Polypeptide
GGGSE

SEQ ID NO: 48 - protein, 5 aa, Artificial Sequence
  Synthetic Polypeptide
GSESG

SEQ ID NO: 49 - protein, 5 aa, Artificial Sequence
  Synthetic Polypeptide
GSEGS

SEQ ID NO: 50 - protein, 35 aa, Artificial Sequence
  Synthetic Polypeptide
GEGGSGEGSSGEGSSSEGGGSEGGGSEGGGSEGGS

SEQ ID NO: 51 - protein, 234 aa, Artificial Sequence
  Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 120 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 58 - protein, 133 aa, Artificial Sequence
  Synthetic polypeptide
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 59 - protein, 182 aa, Artificial Sequence
  Synthetic polypeptide
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 60 - protein, 487 aa, Artificial Sequence
  Synthetic polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 61 - protein, 555 aa, Artificial Sequence
  Synthetic polypeptide
MEWSWVFLFFLSVTTGVHSMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV